(19) United States
(12) Patent Application Publication
(10) Pub. No.: US 2026/0275365 A1
(43) Pub. Date: Sep. 17, 2026

(54) METHODS AND COMPOSITIONS FOR MONOCOT TRANSFORMATION

(71) Applicant: PIONEER HI-BRED INTERNATIONAL, INC., JOHNSTON, IA (US)

(72) Inventors: WILLIAM JAMES GORDON-KAMM, URBANDALE, IA (US); NAGESH SARDESAI, JOHNSTON, IA (US)

(73) Assignee: PIONEER HI-BRED INTERNATIONAL, INC., JOHNSTON, IA (US)

(21) Appl. No.: 19/168,060

(22) PCT Filed: Mar. 22, 2024
(86) PCT No.: PCT/US2024/021129

§ 371 Date: Sep. 23, 2025

Related U.S. Application Data
(60) Provisional application No. 63/492,082, filed on Mar. 24, 2023.

(51) Int. Cl. C12N 15/82; C07K 14/415

(52) U.S. Cl. CPC C12N 15/8201; C07K 14/415; C12N 15/8241; C07K 2319/71

(57) ABSTRACT

Provided herein are polynucleotides encoding recombinant plant transcription factors, the recombinant plant transcription factors, and methods of producing recombinant monocot plants using the recombinant plant transcription factors.

### REFERENCE TO THE SEQUENCE LISTING

The official copy of the sequence listing is submitted electronically and concurrently with this specification. The sequence listing is as an XML formatted file in compliance with the ST26 standard, with a file name of “108740-US-PCT-1 Sequence Listing” created on Sep. 22, 2025 and having a size of 610,725 bytes. The sequence listing contained in this XML file is part of the specification and is herein incorporated by reference in its entirety.

### BACKGROUND

In recent years, there has been a tremendous expansion of the capabilities for the genetic engineering of plants. Current transformation technology provides an opportunity to produce commercially viable transgenic plants, enabling the creation of new plant varieties containing desirable traits. However, there remains a need for plant transformation methods permitting a broader range of transformable and regenerable plant explant tissues and increases in the efficiency of plant transformation methods.

### SUMMARY

The present disclosure describes methods and compositions for transformation of monocot plants. The disclosed methods and compositions greatly improve the frequency of plant transformation (e.g., increasing the number of recombinant TO plants recovered per starting seedling and/or providing TO plants with (1) single-copy (rather than multicopy) insertions of T-DNA containing a gene of interest and (2) no other detectable inserted nucleotide sequences (e.g., from a plasmid backbone).

Provided herein is a polynucleotide encoding a recombinant transcription factor. The recombinant transcription factor comprises a nucleic acid binding domain and a transcriptional activation domain. The nucleic acid binding domain comprises a Bbm truncated polypeptide, and the nucleic acid binding domain is capable of binding a gene regulatory sequence. The transcriptional activation domain comprises a transcriptional activator polypeptide, and the transcriptional activation domain is capable of activating transcription of a target gene. The nucleic acid binding domain comprises at least 50 amino acid residues, and the transcriptional activation domain comprises at least 20 amino acid residues. The nucleic acid binding domain and the transcriptional activation domain do not naturally occur in the same polypeptide.

Also provided herein is a method of producing a recombinant monocot plant. The method comprises contacting a monocot plant cell with a first polynucleotide encoding a gene of interest, wherein the gene of interest is heterologous to the monocot plant cell. The method comprises contacting the monocot plant cell with a second polynucleotide encoding a recombinant transcription factor of the present disclosure. The method comprises selecting a monocot plant cell that has incorporated the gene of interest into its genome and regenerating a recombinant monocot plant from the selected monocot plant cell.

### DETAILED DESCRIPTION

The disclosure is not limited to particular examples, which can, of course, vary. The terminology and exemplary examples used herein are for the purpose of describing aspects of the disclosure only and are not intended to be limiting. As used herein, terms in the singular and the singular forms “a”, “an” and “the”, for example, include plural referents unless the context clearly dictates otherwise. Thus, for example, reference to “plant”, “the plant” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

As used herein, the term “comprising” includes the aspect of “consisting of.”

Unless defined otherwise, numeric ranges recited within the specification are inclusive of the numbers defining the range and include each integer within the defined range. Unless defined otherwise, all technical and scientific terms used herein have the same meaning as commonly understood by one of ordinary skill in the art to which the disclosure pertains.

As used herein, a “recombinant” plant or plant cell comprises a heterologous nucleic acid sequence, a heterologous polypeptide, and/or a heterologous noncoding RNA. A “recombinant” nucleic acid or polypeptide is a nucleic acid or polypeptide which has been altered from its natural form by human intervention via an insertion, deletion, substitution or fusion. The “recombinant” transcription factors of the present disclosure are non-naturally occurring.

As used herein, “heterologous” means that a given nucleic acid sequence has been located in a genome, locus, or construct through human intervention. The heterologous sequence may be naturally occurring, but now located in a genome, locus, or construct where the sequence is not naturally found. Alternatively, the sequence may be “heterologous” in that it is located in a genome, locus, or construct and is non-naturally occurring. A heterologous gene can be inserted into a genome via, for example, transformation and/or site-specific nuclease-based methods.

As used herein, the term “morphogenic gene” means a gene that when ectopically expressed stimulates formation of a somatically-derived structure that can produce a plant. More precisely, ectopic expression, or mutation, or silencing, or decreased expression of the morphogenic gene stimulates the de novo formation of a somatic embryo or an organogenic structure, such as a shoot meristem or an axillary meristem, that can produce a plant or stimulates regeneration of a plant. This stimulated de novo formation occurs either in the cell in which the morphogenic gene is expressed, or silenced, or repressed, or in a neighboring cell. A morphogenic gene can be a transcription factor that regulates expression of other genes, or a gene that influences hormone levels in a plant tissue, both of which can stimulate morphogenic changes. A morphogenic gene may be stably incorporated into the genome of a plant or it may be transiently expressed. In an aspect, expression of the morphogenic gene is controlled. The expression can be controlled transcriptionally or post-transcriptionally. The controlled expression may also be a pulsed expression of the morphogenic gene for a particular period of time. Alternatively, the morphogenic gene may be expressed in only some transformed cells and not expressed in others. The control of expression of the morphogenic gene can be achieved by a variety of methods as disclosed herein below. The morphogenic genes useful in the methods of the present disclosure may be obtained from or derived from any plant species.

As used herein, the term “morphogenic factor” means a morphogenic gene and/or the protein expressed by a morphogenic gene.

A morphogenic gene is involved in plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem or axillary meristem, initiation and/or development of shoots, or a combination thereof, such as WUS/WOX genes (WUS, WUS1, WUS2, WUS3, WOX2A, WOX4, WOX5, or WOX9) see U.S. Pat. Nos. 7,348,468 and 7,256,322 and United States Patent Application publications 20170121722 and 20070271628; Laux et al. (1996) Development 122:87-96; and Mayer et al. (1998) Cell 95:805-815; van der Graaff et al., 2009, Genome Biology 10:248; Dolzblasz et al., 2016, Mol. Plant 19:1028-39 are useful in the methods of the disclosure. Modulation of WUS/WOX is expected to modulate plant and/or plant tissue phenotype including plant metabolism, organ development, stem cell development, cell growth stimulation, organogenesis, regeneration, somatic embryogenesis initiation, accelerated somatic embryo maturation, initiation and/or development of the apical meristem, initiation and/or development of shoot meristem, initiation and/or development of shoots, or a combination thereof. Expression of Arabidopsis WUS can induce stem cells in vegetative tissues, which can differentiate into somatic embryos (Zuo, et al. (2002) Plant J 30:349-359). Also of interest in this regard would be a MYB118 gene (see U.S. Pat. No. 7,148,402), MYB115 gene (see Wang et al. (2008) Cell Research 224-235), a BABYBOOM gene (BBM; see Boutilier et al. (2002) Plant Cell 14:1737-1749), a CLAVATA gene (see, for example, U.S. Pat. No. 7,179,963), an Enhancer of Shoot Regeneration 1 (ESR1) gene (see Banno et al. (2001), The Plant Cell, Vol. 13:2609-2618), a Corngrass1 (Cg1) gene (see Chuck et al. (2007) Nature Genetics, Vol. 39 (4): 544-549), a Cup-Shaped Cotyledon (CUC) gene (see Hibara et al. (2006) The Plant Cell, Vol. 18:2946-2957), a REVOLUTA (REV) gene (see Otsuga et al. (2001) The Plant Journal 25 (2): 223-236), a More Axillary Growth1 (MAX1) gene (see Stirnberg et al. (2002) Development 129:1131-1141), a SUPERSHOOT (SPS) gene (see Tanikanjana, et al. (2001) Genes & Development 15:1577-1588), a Lateral Suppressor (LAS) gene (see Greb et al. (2003) Genes & Development 17:1175-1187), a More Axillary Growth4 (MAX4) gene (see Sorefan et al. (2003) Genes & Development 17:1469-1474), a Stem Cell-Inducing Factor 1 (STEMIN1) gene (see Ishikawa et al. (2019) Nature Plants 5:681-690), a Growth-Regulating Factor 4 (GRF4) gene and/or a GRF-Interacting Factor 1 (GIF1) gene (see Debernardi et al. bioRxiv 2020.08.23.263905; doi: 2020.08.23.263905), and a Growth-Regulating Factor 5 (GRF5) gene (see Kong et al. bioRxiv 2020.08.23.263947; doi: 2020.08.23.263947).

Morphogenic polynucleotide sequences and amino acid sequences of functional WUS/WOX nucleotides/polypeptides can be used in the disclosed methods. As defined herein, a “functional WUS/WOX nucleotide” or “functional WUS/WOX polypeptide” is any polynucleotide encoding a polypeptide, or the peptide itself as the case may be, that contains a homeobox DNA binding domain, a WUS box, and an EAR repressor domain (Ikeda et al., 2009 Plant Cell 21:3493-3505). As demonstrated by Rodriguez et al., (2016 PNAS doi: 1607673113) removal of the dimerization sequence which leaves behind the homeobox DNA binding domain, a WUS box, and an EAR repressor domain results in a functional WUS/WOX polypeptide. The Wuschel protein, designated hereafter as WUS, plays a key role in the initiation and maintenance of the apical meristem, which contains a pool of pluripotent stem cells (Endrizzi, et al., (1996) Plant Journal 10:967-979; Laux, et al., (1996) Development 122:87-96; and Mayer, et al., (1998) Cell 95:805-815). Arabidopsis plants mutant for the WUS gene contain stem cells that are mis-specified and that appear to undergo differentiation. WUS encodes a novel homeodomain protein which presumably functions as a transcriptional regulator (Mayer, et al., (1998) Cell 95:805-815). The stem cell population of Arabidopsis shoot meristems is believed to be maintained by a regulatory loop between the CLAVATA (CLV) genes which promote organ initiation and the WUS gene which is required for stem cell identity, with the CLV genes repressing WUS at the transcript level, and WUS expression being sufficient to induce meristem cell identity and the expression of the stem cell marker CLV3 (Brand, et al., (2000) Science 289:617-619; Schoof, et al., (2000) Cell 100:635-644). Constitutive expression of WUS in Arabidopsis has been shown to lead to adventitious shoot proliferation from leaves (in planta) (Laux, T., Talk Presented at the XVI International Botanical Congress Meeting, Aug. 1-7, 1999, St. Louis, Mo.).

In an aspect, the functional WUS/WOX polypeptides useful in the methods of the present disclosure comprise a WUS, WUS1, WUS2, WUS3, WOX2A, WOX4, WOX5, WOX5A, and/or WOX9 polypeptide (see, U.S. Pat. Nos. 7,348,468 and 7,256,322 and US Patent Application Publication Numbers 2017/0121722 and 2007/0271628, herein incorporated by reference in their entirety and van der Graaff et al., 2009, Genome Biology 10:248). The functional WUS/WOX polypeptides useful in the methods of the present disclosure can be obtained from or derived from any plant including but not limited to monocots, dicots, Angiospermae, and Gymnospermae.

As used herein, “T-DNA” means a portion of a Ti plasmid that is inserted into the genome of a host plant cell.

As used herein, “transformation frequency” refers to a measure of transformation performance in plants that is calculated based on the number of transgenic TO plants recovered per starting seedling. Values over 100% indicate that multiple transgenic TO plants were recovered per starting seedling. Values below 100% indicate that less than 1 transgenic TO plant was recovered per starting seedling.

Recombinant Transcription Factors and Polynucleotides Encoding them

Provided herein is a polynucleotide encoding a recombinant transcription factor. The polynucleotide can comprise any polynucleotide suitable to encode the transcription factor for translation (e.g., protein production). For example, the polynucleotide can comprise a DNA molecule or an RNA molecule. In some examples, the polynucleotide comprises a vector. In some examples, the polynucleotide is within a cell.

As used herein, “vector” refers to a DNA molecule such as a plasmid, cosmid or bacterial phage for introducing a nucleotide construct, for example, an expression cassette or construct, into a host cell. Cloning vectors typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector.

As used herein, “contacting”, “contact”, “contacted”, “comes in contact with” or “in contact with” means “direct contact” or “indirect contact”. For example, cells are placed in a condition where the cells can come into contact with an expression cassette, a nucleotide, a peptide, a RNP (ribonucleoprotein), or other substance disclosed herein. Such expression cassette, nucleotide, peptide, or other substance is allowed to be present in an environment where the cells survive (for example, medium) or expressed in the cell or expressed in an adjacent cell and can act on the cells. For example, a polynucleotide may have direct contact with a cell (e.g., the polynucleotide may be located within a cell) or a polynucleotide may have indirect contact with a cell (e.g., the polynucleotide may be located within a an adjacent cell and expression of the polynucleotide by the adjacent cell may act on the cell). The WUS gene, for example, is known to act on cells via expression originating in adjacent cells. The expression cassettes, polynucleotides, polypeptides, and other substances disclosed herein may contact a cell via T-DNA transfer (e.g., bacteria-mediated transformation), particle bombardment, electroporation, PEG transfection, or RNP (ribonucleoprotein) delivery.

As used herein, the term “expression cassette” means a distinct component of vector DNA consisting of coding and non-coding sequences including 5′ and 3′ regulatory sequences that control expression in a transformed/transfected cell.

As used herein, the term “regulatory sequence” means a segment of a nucleic acid molecule which is capable of increasing or decreasing the expression of a gene. Regulatory sequences include promoters, terminators, enhancer elements, silencing elements, 5′ UTR and 3′ UTR (untranslated regions).

The recombinant transcription factor comprises a nucleic acid binding domain and a transcriptional activation domain. The nucleic acid binding domain is capable of binding a gene regulatory sequence present on a polynucleotide. For example, the nucleic acid binding domain may comprise a portion of a transcription factor that binds nucleic acid regulatory sequences (e.g., those in a promoter operably coupled to a gene's coding sequence).

The nucleic acid binding domain comprises a Bbm truncated polypeptide.

The transcriptional activation domain is capable of causing the activation and/or recruitment of transcriptional machinery (often by binding transcriptional machinery (e.g., an RNA polymerase) or an associated polypeptide). The transcriptional machinery is suitable to transcribe a gene whose ORF is typically 3′ of the regulatory sequence bound by the nucleic acid binding domain. Thus, the recombinant transcription factor comprises two domains which function to (1) bind (usually upstream) of a regulated gene (via the nucleic acid binding domain) and (2) activate transcription of a target gene (e.g., the regulated gene) via the transcriptional activation domain when positioned suitably by the nucleic acid binding domain.

The transcriptional activation domain comprises a transcriptional activator polypeptide.

The nucleic acid binding domain comprises at least 50 amino acid residues. In some examples, the nucleic acid binding domain comprises at least 55 amino acid residues, at least 60 amino acid residues, at least 65 amino acid residues, at least 70 amino acid residues, at least 80 amino acid residues, at least 90 amino acid residues, or at least 100 amino acid residues.

The transcriptional activation domain and/or the transcriptional activator polypeptide comprises at least 20 amino acid residues. In some examples, the transcriptional activation domain comprises at least 25 amino acid residues, at least 30 amino acid residues, at least 35 amino acid residues, at least 40 amino acid residues, or at least 50 amino acid residues.

The nucleic acid binding domain and the transcriptional activation domain do not naturally occur in the same polypeptide.

### Nucleic Acid Binding Domain

The Bbm truncated polypeptide comprises a truncated Baby Boom (Bbm) subfamily polypeptide. The Bbm subfamily of polypeptides is a subset of the AP2 family of plant transcription factors. The Bbm subfamily comprises Bbm, Bbm1, and Bbm2 plant genes from various different plant species which comprise some or all of the Bbm, Bbm1, and Bbm2 genes. It is noted that the Bbm gene from Zea mays (which has the polypeptide sequence of SEQ ID NO: 15 and cDNA sequence of SEQ ID NO: 14 and for which the subfamily is named) was initially termed “Odp2” when the gene was discovered. Accordingly, when “Bbm” or “Odp2” are used herein both terms refer to the gene displaying SEQ ID NOs: 14 and 15 in Zea mays, unless context dictates otherwise. Many related Bbm subfamily genes exist in other plants and in Zea mays (e.g., Zea mays BBM2). Example polypeptide sequences from such genes are disclosed in SEQ ID NOs: 15, 22, 26, 30, 34, 38, 42, 46, 50, 54, 58, 62, 66, 70, 74, 78, 82, 86, 90, 94, 98, 102, 106, 110, 114, 118, and 122. All of these polypeptide sequences can be truncated to form a Bbm truncated polypeptide as disclosed herein and as shown in the sequence listing.

The nucleic acid binding domain of the recombinant transcription factor comprises a Bbm truncated polypeptide. A “Bbm truncated polypeptide” as used herein is a fragment of a Bbm, Bbm1, or Bbm2 polypeptide. The Bbm truncated polypeptide also retains nucleic acid binding activity sufficient to activate genes (via the transcriptional activation domain) controlled by the regulatory sequence bound by the Bbm truncated polypeptide. It was surprisingly discovered that making such truncations can improve the performance of the Bbm morphogenic gene during monocot transformation, as described in the examples.

For example, the Bbm truncated polypeptide can comprise at least 90% sequence identity to any one of SEQ ID NOs: 21, 25, 29, 33, 37, 41, 45, 49, 53, 57, 61, 65, 69, 73, 77, 81, 85, 89, 93, 97, 101, 105, 109, 113, 117, 121, or 125.

For example, the Bbm truncated polypeptide can comprise at least 91% sequence identity to any one of SEQ ID NOs: 21, 25, 29, 33, 37, 41, 45, 49, 53, 57, 61, 65, 69, 73, 77, 81, 85, 89, 93, 97, 101, 105, 109, 113, 117, 121, or 125.

For example, the Bbm truncated polypeptide can comprise at least 92% sequence identity to any one of SEQ ID NOs: 21, 25, 29, 33, 37, 41, 45, 49, 53, 57, 61, 65, 69, 73, 77, 81, 85, 89, 93, 97, 101, 105, 109, 113, 117, 121, or 125.

For example, the Bbm truncated polypeptide can comprise at least 93% sequence identity to any one of SEQ ID NOs: 21, 25, 29, 33, 37, 41, 45, 49, 53, 57, 61, 65, 69, 73, 77, 81, 85, 89, 93, 97, 101, 105, 109, 113, 117, 121, or 125.

For example, the Bbm truncated polypeptide can comprise at least 94% sequence identity to any one of SEQ ID NOs: 21, 25, 29, 33, 37, 41, 45, 49, 53, 57, 61, 65, 69, 73, 77, 81, 85, 89, 93, 97, 101, 105, 109, 113, 117, 121, or 125.

For example, the Bbm truncated polypeptide can comprise at least 95% sequence identity to any one of SEQ ID NOs: 21, 25, 29, 33, 37, 41, 45, 49, 53, 57, 61, 65, 69, 73, 77, 81, 85, 89, 93, 97, 101, 105, 109, 113, 117, 121, or 125.

For example, the Bbm truncated polypeptide can comprise at least 96% sequence identity to any one of SEQ ID NOs: 21, 25, 29, 33, 37, 41, 45, 49, 53, 57, 61, 65, 69, 73, 77, 81, 85, 89, 93, 97, 101, 105, 109, 113, 117, 121, or 125.

For example, the Bbm truncated polypeptide can comprise at least 97% sequence identity to any one of SEQ ID NOs: 21, 25, 29, 33, 37, 41, 45, 49, 53, 57, 61, 65, 69, 73, 77, 81, 85, 89, 93, 97, 101, 105, 109, 113, 117, 121, or 125.

For example, the Bbm truncated polypeptide can comprise at least 98% sequence identity to any one of SEQ ID NOs: 21, 25, 29, 33, 37, 41, 45, 49, 53, 57, 61, 65, 69, 73, 77, 81, 85, 89, 93, 97, 101, 105, 109, 113, 117, 121, or 125.

For example, the Bbm truncated polypeptide can comprise at least 99% sequence identity to any one of SEQ ID NOs: 21, 25, 29, 33, 37, 41, 45, 49, 53, 57, 61, 65, 69, 73, 77, 81, 85, 89, 93, 97, 101, 105, 109, 113, 117, 121, or 125.

In some examples, the Bbm truncated polypeptide can comprise the sequence of any one of SEQ ID NOs: 21, 25, 29, 33, 37, 41, 45, 49, 53, 57, 61, 65, 69, 73, 77, 81, 85, 89, 93, 97, 101, 105, 109, 113, 117, 121, or 125.

In some examples, the nucleic acid binding domain comprises a Bbm truncated polypeptide and optionally further comprises and is operably linked to: a Bbm A polypeptide, a Bbm B polypeptide, or both a Bbm A polypeptide and a Bbm B polypeptide. The Bbm truncated polypeptide can be present in the nucleic acid binding domain without a Bbm A polypeptide and without a Bbm B polypeptide. Alternatively, the Bbm truncated polypeptide can be present in the nucleic acid binding domain along with a Bbm A polypeptide, a Bbm B polypeptide, or both. In examples comprising a Bbm A polypeptide and/or a Bbm B polypeptide, Bbm A polypeptides and Bbm B polypeptides are arranged N-terminal to the Bbm truncated polypeptide. If both a Bbm B polypeptide and a Bbm A polypeptide are present, the Bbm B polypeptide is arranged immediately N-terminal to the Bbm A polypeptide. Accordingly, the nucleic acid binding domain can comprise any of the following exemplary configurations:

The nucleic acid binding domain can comprise: Bbm truncated polypeptide (e.g., SEQ ID NO: 21).

The nucleic acid binding domain can comprise: Bbm B polypeptide-Bbm truncated polypeptide (e.g., SEQ ID NO: 17 and 21).

The nucleic acid binding domain can comprise: Bbm A polypeptide-Bbm truncated polypeptide (e.g., SEQ ID NO: 19 and 21).

The nucleic acid binding domain can comprise: Bbm B polypeptide-Bbm A polypeptide-Bbm truncated polypeptide (e.g., SEQ ID NO: 17, 19, and 21).

The nucleic acid binding domain can comprise: Bbm B polypeptide-Bbm A polypeptide-Bbm B polypeptide-Bbm A polypeptide-Bbm truncated polypeptide (e.g., SEQ ID NO: 17, 19, 17, 19, and 21).

In some examples, a linker peptide can optionally be located immediately between any of the three components if more than the Bbm truncated polypeptide is used. (E.g., SEQ ID NO: 17-linker-SEQ ID NO: 19-linker-SEQ ID NO: 21).

In some examples, the Bbm B polypeptide can comprise the amino acid sequence of any one of SEQ ID NO: 17, 23, 27, 31, 35, 39, 43, 47, 51, 55, 59, 63, 67, 71, 75, 79, 83, 87, 91, 95, 99, 103, 107, 111, 115, 119, or 123.

In some examples, the Bbm B polypeptide can comprise the amino acid sequence of any one of SEQ ID NO: 17, 23, 27, 31, 35, 39, 43, 47, 51, 55, 59, 63, 67, 71, 75, 79, 83, 87, 91, 95, 99, 103, 107, 111, 115, 119, or 123 or any otherwise identical sequence where a single amino acid substitution, insertion, or deletion has been made.

In some examples, the Bbm A polypeptide can comprise the amino acid sequence of any one of SEQ ID NO: 19, 24, 28, 32, 36, 40, 44, 48, 52, 56, 60, 64, 68, 72, 76, 80, 84, 88, 92, 96, 100, 104, 108, 112, 116, 120, or 124.

In some examples, the Bbm A polypeptide can comprise the amino acid sequence of any one of SEQ ID NO: 19, 24, 28, 32, 36, 40, 44, 48, 52, 56, 60, 64, 68, 72, 76, 80, 84, 88, 92, 96, 100, 104, 108, 112, 116, 120, or 124 or any otherwise identical sequence where a single amino acid substitution, insertion, or deletion has been made.

### Transcriptional Activation Domain

In recombinant transcription factors of the present disclosure, the transcriptional activation domain comprises a transcriptional activator polypeptide.

The transcriptional activator polypeptide can comprise a member of the AP2/ERF super-family which includes the sub-groups: the ERF/DREB family, the AP2 family, and the RAV family. The ERF/DREB family is the largest of these families containing, for example, 122 members in Arabidopsis which can be further split into sub-groups of proteins that respond to ethylene (ERF genes), dehydration (DREB genes), and proteins that contain C-repeat binding factors (CBF genes). While these sub-groups of AP2/ERF proteins are typically categorized based on their binding to canonical DNA sequences, specific family members also contain repressor elements (such as the EAR repressor motif characterized by Ohta M et al., 2001, Plant Cell 13:1959-1968), while other AP2/ERF members contain activation motifs such as the well-characterized EDLL peptide located within the C-terminal domain of ATERF98 protein (Tiwari S B et al 2012, Plant J 70:855-865).

Examples of transcriptional activator protein families include examples such as Dof proteins such as maize DOF1 (Yanagisawa S, 2001, Plant Cell Physiol. 42:813-822), C-repeat DRE-binding factors such as CBF1 (Achard P et al., 2008, Plant Cell 20:2117-2129), proteins containing drought responsive elements such as DREB1 (Maruyama K et al., 2004, Plant Journal 38:982-993), ethylene response factor proteins such as ERF1 (Fujimoto S Y et al., 2000, Plant Cell 12:393-404) or ERF2 (Nakano T et al., 2006, Plant Cell Physiol. 47:554-558), proteins with the octadecanoid-derivative responsive Catharanthus AP2 domain such as ORCA (Menke F L H et al., 1999, EMJO J 18:4455-4463), and Pseudomonas syringae cv Tomato interacting-associated proteins such as PIT1 (Gu Y-Q et al., 2002, Plant Cell 14:817-831).

In some examples, the transcriptional activator polypeptide comprises at least 90% amino acid sequence identity to any one of SEQ ID NO: 127, 129, 131, 133, 135, 137-158, 160, or 164-166.

In some examples, the transcriptional activator polypeptide comprises at least 91% amino acid sequence identity to any one of SEQ ID NO: 127, 129, 131, 133, 135, 137-158, 160, or 164-166.

In some examples, the transcriptional activator polypeptide comprises at least 92% amino acid sequence identity to any one of SEQ ID NO: 127, 129, 131, 133, 135, 137-158, 160, or 164-166.

In some examples, the transcriptional activator polypeptide comprises at least 93% amino acid sequence identity to any one of SEQ ID NO: 127, 129, 131, 133, 135, 137-158, 160, or 164-166.

In some examples, the transcriptional activator polypeptide comprises at least 94% amino acid sequence identity to any one of SEQ ID NO: 127, 129, 131, 133, 135, 137-158, 160, or 164-166.

In some examples, the transcriptional activator polypeptide comprises at least 95% amino acid sequence identity to any one of SEQ ID NO: 127, 129, 131, 133, 135, 137-158, 160, or 164-166.

In some examples, the transcriptional activator polypeptide comprises at least 96% amino acid sequence identity to any one of SEQ ID NO: 127, 129, 131, 133, 135, 137-158, 160, or 164-166.

In some examples, the transcriptional activator polypeptide comprises at least 97% amino acid sequence identity to any one of SEQ ID NO: 127, 129, 131, 133, 135, 137-158, 160, or 164-166.

In some examples, the transcriptional activator polypeptide comprises at least 98% amino acid sequence identity to any one of SEQ ID NO: 127, 129, 131, 133, 135, 137-158, 160, or 164-166.

In some examples, the transcriptional activator polypeptide comprises at least 99% amino acid sequence identity to any one of SEQ ID NO: 127, 129, 131, 133, 135, 137-158, 160, or 164-166.

In some examples, the transcriptional activator polypeptide comprises the sequence of any one of SEQ ID NO: 127, 129, 131, 133, 135, 137-158, 160, or 164-166.

In some examples, the transcriptional activator polypeptide comprises a CBF1A polypeptide. In some examples, the CBF1A polypeptide comprises at least 90%, 91%, 92%, 93%, 94%, 95%, 96%, 97%, 98%, or 99% amino acid sequence identity to SEQ ID NO: 127. In some examples, the CBF1A polypeptide comprises the sequence of SEQ ID NO: 127.

In some examples, the transcriptional activator polypeptide comprises a CBF31 polypeptide. In some examples, the CBF3I polypeptide comprises at least 90%, 91%, 92%, 93%, 94%, 95%, 96%, 97%, 98%, or 99% amino acid sequence identity to SEQ ID NO: 129. In some examples, the CBF3I polypeptide comprises the sequence of SEQ ID NO: 129.

In some examples, the transcriptional activation domain comprises multiple transcriptional activator polypeptides. For example, the transcriptional activation domain can comprise two CBF1A polypeptides, two CBF3I polypeptides, or one CBF1A polypeptide and one CBF3I polypeptide. In some examples, the transcriptional activation domain comprises more than one transcriptional activator polypeptide, with each independently comprising a sequence selected from: SEQ ID NO: 127, 129, 131, 133, 135, 137-158, 160, or 164-166.

In some examples, a recombinant transcription factor of the present disclosure can comprise a nucleic acid binding domain comprising a Bbm B polypeptide, a Bbm A polypeptide, and a Bbm truncated polypeptide and a transcriptional activator polypeptide comprising a CBF1A polypeptide. In specific examples, the Bbm B polypeptide comprises SEQ ID NO: 17, the Bbm A polypeptide comprises SEQ ID NO: 19, the Bbm truncated polypeptide comprises at least 90%, 91%, 92%, 93%, 94%, 95%, 96%, 97%, 98%, 99%, or 100% amino acid sequence identity amino acid sequence identity to SEQ ID NO: 21, and the CBF1A polypeptide comprises at least 90%, 91%, 92%, 93%, 94%, 95%, 96%, 97%, 98%, 99%, or 100% amino acid sequence identity amino acid sequence identity to SEQ ID NO: 127. More specifically, the recombinant transcription factor can be arranged as Nterm-B-A-Bbm404-[transcriptional activator domain]-Cterm or as Nterm-B-A-Bbm404-CBF1A-Cterm.

In some examples, a recombinant transcription factor of the present disclosure can comprise a nucleic acid binding domain comprising a Bbm truncated polypeptide and transcriptional activation domain comprising a CBF1A polypeptide. In specific examples, the Bbm truncated polypeptide comprises at least 90%, 91%, 92%, 93%, 94%, 95%, 96%, 97%, 98%, 99%, or 100% amino acid sequence identity to SEQ ID NO: 21, and the CBF1A polypeptide comprises at least 90%, 91%, 92%, 93%, 94%, 95%, 96%, 97%, 98%, 99%, or 100% amino acid sequence identity to SEQ ID NO: 127.

### Methods of Producing Recombinant Plants

Provided herein is a method of producing a recombinant monocot plant. The method comprises contacting a monocot plant cell with a first polynucleotide encoding a gene of interest (the gene of interest is heterologous to the monocot plant cell), contacting the monocot plant cell with a second polynucleotide encoding a recombinant transcription factor of the present disclosure, selecting a monocot plant cell that has incorporated the gene of interest into its genome, and regenerating a recombinant monocot plant from the selected monocot plant cell.

Contacting the monocot plant cell(s) with one or both of the first and second polynucleotides can comprise transformation methods. Transformation methods can comprise bacteria-mediated and/or biolistic-mediated gene transfer, in addition to electroporation, PEG transfection, or RNP (ribonucleoprotein) delivery to produce regenerable plant cells having an incorporated nucleotide sequence of interest. Bacterial strains useful in the methods of the disclosure include, but are not limited to, a disarmed Agrobacterium, an Ochrobactrum bacteria or a Rhizobiaceae bacteria. Disarmed Agrobacteria useful in the present methods include, but are not limited to, AGL-1, EHA105, GV3101, LBA4404, LBA4404 THY—(see U.S. Pat. No. 8,334,429 incorporated herein by reference in its entirety) and LBA4404 TD THY—in which both copies of the Tn904 transposon removed have been removed from LBA4404 THY—(see PCT/US20/24993 filed Mar. 26, 2020 which claims the benefit of U.S. Provisional Patent Application No. 62/825,054 filed on Mar. 28, 2019, all of which is hereby incorporated herein in its entirety by reference). Agrobacterium strain LBA4404 TD THY—is A. tumefaciens LBA4404 THY-strain deposited with the ATCC, assigned Accession Number PTA-10531 wherein a functional Tn904 transposon is not present or both copies of the Tn904 transposon have been deleted. Ochrobactrum bacterial strains useful in the present methods include, but are not limited to, those disclosed in U.S. Pat. Pub. No. US20180216123 incorporated herein by reference in its entirety. Rhizobiaceae bacterial strains useful in the present methods include, but are not limited to, those disclosed in U.S. Pat. No. 9,365,859 incorporated herein by reference in its entirety.

There are a variety of methods for regenerating plants from plant tissue/cells. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In: Methods for Plant Molecular Biology, (Eds.), Academic Press, Inc., San Diego, Calif., herein incorporated by reference in its entirety). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants.

In some examples, the method of producing a recombinant monocot plant can comprises contacting the monocot plant cell with a third polynucleotide encoding a functional Wuschel or Wuschel homeobox (WUS/WOX) polypeptide.

In other examples, the method does not comprise contacting the monocot plant cell with a third polynucleotide encoding a functional WUS/WOX polypeptide.

In some examples, the monocot plant cell comprises a Poaceae plant cell. In certain examples, the monocot plant cell comprises a plant cell of any one of the following species: Zea mays, Oryza sativa, Triticum aestivum, Setaria italica, Hordeum vulgare, Cenchrus americanus, Saccharum officinarum, or Sorghum bicolor.

In some examples, contacting cells with any combination of the three polynucleotides comprises bacteria-mediated transformation or particle bombardment.

In some examples, the first polynucleotide is present on a first vector, and the second polynucleotide is present on a second vector. In some examples, the second vector further comprises the third polynucleotide. In other examples, each polynucleotide is provided on a vector that does not comprise either of the other two polynucleotides.

In some examples, the monocot plant cell is an immature embryo cell or a leaf cell.

In some examples, the gene of interest comprises a trait gene, a site-specific endonuclease, a guide polynucleotide, or a combination thereof. Numerous trait genes are known in the art and can be used in the methods disclosed herein. By way of illustration, without intending to be limiting, trait genes that confer resistance to insects or diseases, trait genes that confer resistance to a herbicide, trait genes that confer or contribute to an altered grain characteristic, such as altered fatty acids, altered phosphorus content, altered carbohydrates or carbohydrate composition, altered antioxidant content or composition, or altered essential seed amino acids content or composition are examples of the types of trait genes which can be operably linked to a promoter for expression in plants transformed by the methods disclosed herein. Additional genes known in the art may be included in the expression cassettes useful in the methods disclosed herein. Non-limiting examples include genes that create a site for site specific DNA integration, genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress, or other genes and transcription factors that affect plant growth and agronomic traits such as yield, flowering, plant growth and/or plant structure.

A site-specific endonuclease refers to a polypeptide that is capable of cutting DNA (e.g., genomic DNA) at specific location based on its affinity for a specific DNA sequence and/or base pairing by a guide polynucleotide that complexed with the site-specific endonuclease. Examples include, but are not limited to, Cas9 (complexed with a guide RNA) and zinc finger nucleases.

In some examples, the method comprises excising one or both of the second and third polynucleotides from the genome of the selected cell. In some examples, examples, the third polynucleotide is not employed and, therefore, not excised.

The present disclosure will be more fully understood by reference to the following clauses.
1. A polynucleotide encoding a recombinant transcription factor, the recombinant transcription factor comprising a nucleic acid binding domain and a transcriptional activation domain,
   wherein the nucleic acid binding domain comprises a Bbm truncated polypeptide, and the nucleic acid binding domain is capable of binding a gene regulatory sequence;
   wherein the transcriptional activation domain comprises a transcriptional activator polypeptide, wherein the transcriptional activation domain is capable of activating transcription of a target gene;
   wherein the nucleic acid binding domain comprises at least 50 amino acid residues, and the transcriptional activation domain comprises at least 20 amino acid residues; and
   wherein the nucleic acid binding domain and the transcriptional activation domain do not naturally occur in the same polypeptide.
2. The polynucleotide of clause 1, wherein the nucleic acid binding domain comprises a Bbm truncated polypeptide operably linked to: (i) a Bbm A polypeptide, (ii) a Bbm B polypeptide, or (iii) both a Bbm A polypeptide and a Bbm B polypeptide.
3. The polynucleotide of clause 1, wherein the nucleic acid binding domain does not comprise a Bbm A polypeptide or a Bbm B polypeptide.
4. The polynucleotide of any one of clauses 1-3, wherein the Bbm truncated polypeptide comprises at least 90% amino acid sequence identity to any one of SEQ ID NO: 21, 25, 29, 33, 37, 41, 45, 49, 53, 57, 61, 65, 69, 73, 77, 81, 85, 89, 93, 97, 101, 105, 109, 113, 117, 121, or 125.
5. The polynucleotide of any one of clauses 2 or 4, wherein the Bbm B polypeptide comprises any one of SEQ ID NO: 17, 23, 27, 31, 35, 39, 43, 47, 51, 55, 59, 63, 67, 71, 75, 79, 83, 87, 91, 95, 99, 103, 107, 111, 115, 119, or 123 or any otherwise identical sequence where a single amino acid substitution, insertion, or deletion has been made.
6. The polynucleotide of any one of clauses 2, 4, or 5, wherein the Bbm A polypeptide comprises any one of SEQ ID NO: 19, 24, 28, 32, 36, 40, 44, 48, 52, 56, 60, 64, 68, 72, 76, 80, 84, 88, 92, 96, 100, 104, 108, 112, 116, 120, or 124 or any otherwise identical sequence where a single amino acid substitution, insertion, or deletion has been made.
7. The polynucleotide of any one of clauses 1-6, wherein the transcriptional activator polypeptide comprises at least 90% amino acid sequence identity to any one of SEQ ID NO: 127, 129, 131, 133, 135, 137-158, 160, or 164-166.
8. The polynucleotide of any one of clauses 1-7, wherein the transcriptional activator polypeptide comprises a CBF1A polypeptide.
9. The polynucleotide of any one of clauses 1-8, wherein the transcriptional activator polypeptide comprises at least 90%, 91%, 92%, 93%, 94%, 95%, 96%, 97%, 98%, 99%, or 100% amino acid sequence identity to SEQ ID NO: 127.
10. The polynucleotide of any one of clauses 1-9, wherein the transcriptional activator polypeptide comprises a CBF3I polypeptide.
11. The polynucleotide of any one of clauses 1-10, wherein the transcriptional activator polypeptide comprises at least 90%, 91%, 92%, 93%, 94%, 95%, 96%, 97%, 98%, 99%, or 100% amino acid sequence identity to SEQ ID NO: 129.
12. The polynucleotide of any one of clauses 1-11, wherein the transcriptional activation domain comprises multiple transcriptional activator polypeptides.
13. The polynucleotide of clause 12, wherein the transcriptional activation domain comprises two CBF1A polypeptides, two CBF3I polypeptides, or one CBF1A polypeptide and one CBF31 polypeptide.
14. The polynucleotide of any one of clauses 1, 2, or 4-13, wherein the nucleic acid binding domain comprises a Bbm B polypeptide, a Bbm A polypeptide, and a Bbm truncated polypeptide and
   wherein the transcriptional activator polypeptide comprises a CBF1A polypeptide.
15. The polynucleotide of clause 14, wherein the Bbm B polypeptide comprises SEQ ID NO: 17, the Bbm A polypeptide comprises SEQ ID NO: 19, and the Bbm truncated polypeptide comprises at least 90% amino acid sequence identity to SEQ ID NO: 21 and
   wherein the CBF1A polypeptide comprises at least 90% amino acid sequence identity to SEQ ID NO: 127.
16. The polynucleotide of any one of clauses 1, 3, 4 or 7-13, wherein the nucleic acid binding domain comprises a Bbm truncated polypeptide and wherein the transcriptional activator polypeptide comprises a CBF1A polypeptide.
17. The polynucleotide of clause 16, wherein the Bbm truncated polypeptide comprises at least 90% amino acid sequence identity to SEQ ID NO: 21 and
   wherein the CBF1A polypeptide comprises at least 90% amino acid sequence identity to SEQ ID NO: 127.
18. The polynucleotide of any one of clauses 1-17, wherein the Bbm truncated polypeptide comprises at least 90%, 91%, 92%, 93%, 94%, 95%, 96%, 97%, 98%, 99%, or 100% amino acid sequence identity to SEQ ID NO: 21.
19. The polynucleotide of any one of clauses 1-18, wherein the Bbm truncated polypeptide is encoded by a polynucleotide comprising at least 90%, 91%, 92%, 93%, 94%, 95%, 96%, 97%, 98%, 99%, or 100% nucleotide sequence identity to SEQ ID NO: 20.
20. The polynucleotide of any one of clauses 1, 2, or 4-19, wherein the Bbm B polypeptide comprises at least 90%, 91%, 92%, 93%, 94%, 95%, 96%, 97%, 98%, 99%, or 100% amino acid sequence identity to SEQ ID NO: 17.
21. The polynucleotide of any one of clauses 1, 2, or 4-20, wherein the Bbm B polypeptide is encoded by a polynucleotide comprising at least 90%, 91%, 92%, 93%, 94%, 95%, 96%, 97%, 98%, 99%, or 100% nucleotide sequence identity to SEQ ID NO: 16.
22. The polynucleotide of any one of clauses 1, 2, or 4-21, wherein the Bbm A polypeptide comprises at least 90%, 91%, 92%, 93%, 94%, 95%, 96%, 97%, 98%, 99%, or 100% amino acid sequence identity to SEQ ID NO: 19.
23. The polynucleotide of any one of clauses 1, 2, or 4-22, wherein the Bbm A polypeptide is encoded by a polynucleotide comprising at least 90%, 91%, 92%, 93%, 94%, 95%, 96%, 97%, 98%, 99%, or 100% nucleotide sequence identity to SEQ ID NO: 18.
24. The polynucleotide of any one of clauses 8-23, wherein the CBF1A polypeptide is encoded by a polynucleotide comprising a nucleotide sequence comprising at least 90%, 91%, 92%, 93%, 94%, 95%, 96%, 97%, 98%, 99%, or 100% identity to SEQ ID NO: 126.
25. The polynucleotide of any one of clauses 10-24, wherein the CBF3I polypeptide is encoded by a polynucleotide comprising at least 90%, 91%, 92%, 93%, 94%, 95%, 96%, 97%, 98%, 99%, or 100% identity to the sequence of SEQ ID NO: 128.
26. The recombinant transcription factor encoded by the polynucleotide of any one of clauses 1-25.
27. A method of producing a recombinant monocot plant, the method comprising:
   contacting a monocot plant cell with a first polynucleotide encoding a gene of interest, wherein the gene of interest is heterologous to the monocot plant cell;
   contacting the monocot plant cell with a second polynucleotide encoding the recombinant transcription factor of clause 26;
   selecting a monocot plant cell that has incorporated the gene of interest into its genome; and
   regenerating a recombinant monocot plant from the selected monocot plant cell.
28. The method of clause 27, further comprising contacting the monocot plant cell with a third polynucleotide encoding a functional Wuschel or Wuschel homeobox (WUS/WOX) polypeptide.
29. The method of clause 27, wherein the method does not comprise contacting the monocot plant cell with a third polynucleotide encoding a functional WUS/WOX polypeptide.
30. The method of any one of clauses 27-29, wherein the monocot plant cell comprises a Poaceae plant cell.
31. The method of any one of clauses 27-30, wherein the monocot plant cell comprises a plant cell of any one of the following species: Zea mays, Oryza sativa, Triticum aestivum, Setaria italica, Hordeum vulgare, Cenchrus americanus, Saccharum officinarum, or Sorghum bicolor.
32. The method of any one of clauses 27-31, wherein the contacting steps comprise bacteria-mediated transformation or particle bombardment.
33. The method of any one of clauses 27-32, wherein the first polynucleotide is present on a first vector, and the second polynucleotide is present on a second vector.
34. The method of clause 33, wherein the second vector further comprises the third polynucleotide.
35. The method of any one of clauses 27-34, wherein the monocot plant cell is an immature embryo cell or a leaf cell.
36. The method of any one of clauses 27-35, wherein the gene of interest comprises a trait gene, a site-specific endonuclease, a guide polynucleotide, or a combination thereof.
37. The method of any one of clauses 27-36, further comprising excising one or both of the second and third polynucleotides from the genome of the selected cell.

Examples—The present disclosure will be more fully appreciated with reference to the following non-limiting examples.

### Example 1: Sequences

Sequences useful in the methods of the disclosure are presented in Table 1 and provided in the sequence listing.

TABLE 1

| | Polynucleotide | | |
|---|---|---|---|
| | (DNA) or | | |
| | Polypeptide | | |
| SEQ ID NO: | (PRT) | NAME | DESCRIPTION |
| 1 | DNA | PHP71539 | VIRB1 + VIRB2 + VIRB3 + VIRB4 + |
| | | | VIRB5 + VIRB6 + VIRB7 + VIRB8 + |
| | | | VIRB9 + VIRB10 + VIRB11 + VIRG + |
| | | | VIRC2 + VIRC1 + VIRD1 + VIRD2 + |
| | | | VIRD3 + VIRD4 + VIRD5 + VIRE1 + |
| | | | VIRE2 + VIRE3 + GENT + COLE1 ORI + |
| | | | PVS1 ORI |
| 2 | DNA | PHP96037 | Entire plasmid sequence |
| 3 | DNA | PHP96037 | RB + LOXP + NOS PRO::ZM- |
| | | | WUS2::IN2-1 TERM + FMV |
| | | | ENH::PCSV ENH::MMV ENH::UBI1ZM |
| | | | PRO::UBI1ZM 5UTR::UBI1ZM |
| | | | INTRON1::ZM-ODP2::OS-T28 TERM + |
| | | | AT-5-IV-2 INS + ZM-HSP17.7 |
| | | | PRO::MO-CRE-EXON1::ST-LS1 |
| | | | INTRON1::MO-CRE EXON2::PINII |
| | | | TERM + AT-5-IV-2 INS + LOXP + SB- |
| | | | UBI PRO::SB-UBI INTRON1::ZS- |
| | | | GREEN1::OS-UBI TERM + SB-ALS |
| | | | PRO::ZM-ALS (HRA)::SB-PEPC1 |
| | | | TERM + LB |
| 4 | DNA | PHP97334 | RB + LOXP + NOS PRO::ZM- |
| | | | WUS2::IN2-1 TERM + FMV |
| | | | ENH::PCSV ENH::MMV ENH::UBI1ZM |
| | | | PRO::UBI1ZM 5UTR::UBI1ZM |
| | | | INTRON1::ZM-ODP2::OS-T28 TERM + |
| | | | AT-5-IV-2 INS + ZM-HSP17.7 |
| | | | PRO::MO-CRE-EXON1::ST-LS1 |
| | | | INTRON1::MO-CRE EXON2::PINII |
| | | | TERM + AT-5-IV-2 INS + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1::NPTII::SB- |
| | | | UBI TERM + LB |
| 5 | DNA | RV045471 = | RB + LOXP + NOS PRO::ZM- |
| | | PHP102072 | WUS2::IN2-1 TERM + FMV |
| | | | ENH::PCSV ENH::MMV ENH::UBI1ZM |
| | | | PRO::UBI1ZM 5UTR::UBI1ZM |
| | | | INTRON1::ZM-BBM404::OS-T28 |
| | | | TERM + AT-5-IV-2 INS + ZM-HSP17.7 |
| | | | PRO::MO-CRE-EXON1::ST-LS1 |
| | | | INTRON1::MO-CRE EXON2::PINII |
| | | | TERM + OS-UBI TERM + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1::NPTII::SB- |
| | | | UBI TERM + LB |
| 6 | DNA | RV045472 = | RB + LOXP + NOS PRO::ZM- |
| | | PHP101977 | WUS2::IN2-1 TERM + FMV |
| | | | ENH::PCSV ENH::MMV ENH::UBI1ZM |
| | | | PRO::UBI1ZM 5UTR::UBI1ZM |
| | | | INTRON1::ZM-B:A:BBM404::OS-T28 |
| | | | TERM + AT-5-IV-2 INS + ZM-HSP17.7 |
| | | | PRO::MO-CRE-EXON1::ST-LS1 |
| | | | INTRON1::MO-CRE EXON2::PINII |
| | | | TERM + OS-UBI TERM + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1::NPTII::SB- |
| | | | UBI TERM + LB |
| 7 | DNA | RV045473 = | RB + LOXP + NOS PRO::ZM- |
| | | PHP103858 | WUS2::IN2-1 TERM + FMV |
| | | | ENH::PCSV ENH::MMV ENH::UBI1ZM |
| | | | PRO::UBI1ZM 5UTR::UBI1ZM |
| | | | INTRON1::ZM-B:A:B:A:BBM404::OS- |
| | | | T28 TERM + AT-5-IV-2 INS + ZM- |
| | | | HSP17.7 PRO::MO-CRE-EXON1::ST- |
| | | | LS1 INTRON1::MO-CRE EXON2::PINII |
| | | | TERM + OS-UBI TERM + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1:NPTII::SB- |
| | | | UBI TERM + LB |
| 8 | DNA | RV045474 = | RB + LOXP + NOS PRO::ZM- |
| | | PHP101978 | WUS2::IN2-1 TERM + FMV |
| | | | ENH::PCSV ENH::MMV ENH::UBI1ZM |
| | | | PRO::UBI1ZM 5UTR::UBI1ZM |
| | | | INTRON1::ZM-B:A:BBM404:PROTEIN |
| | | | LINKER 1:AT-CBF1A (MO):OS-T28 |
| | | | TERM + AT-5-IV-2 INS + ZM-HSP17.7 |
| | | | PRO::MO-CRE-EXON1::ST-LS1 |
| | | | INTRON1::MO-CRE EXON2::PINII |
| | | | TERM + OS-UBI TERM + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1::NPTII::SB- |
| | | | UBI TERM + LB |
| 9 | DNA | RV048557 = | RB + LOXP + OS-ACTIN PRO::ZM- |
| | | PHP104187 | WUS2::IN2-1 TERM + FMV |
| | | | ENH::PCSV ENH::MMV ENH::UBI1ZM |
| | | | PRO::UBI1ZM 5UTR::UBI1ZM |
| | | | INTRON1::ZM-B:A:BBM404:PROTEIN |
| | | | LINKER 1:AT-CBF1A (MO):OS-T28 |
| | | | TERM + AT-5-IV-2 INS + ZM-HSP17.7 |
| | | | PRO::MO-CRE-EXON1::ST-LS1 |
| | | | INTRON1::MO-CRE EXON2::PINII |
| | | | TERM + OS-UBI TERM + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1::NPTII::SB- |
| | | | UBI TERM + LB |
| 10 | DNA | RV048363 = | RB + LOXP + NOS PRO::ZM- |
| | | PHP103910 | WUS2::IN2-1 TERM + UBI1ZM |
| | | | PRO::UBI1ZM 5UTR::UBI1ZM |
| | | | INTRON1::ZM-BBM:PROTEIN LINKER |
| | | | 1:AT-CBF1A (MO):OS-T28 TERM + |
| | | | AT-5-IV-2 INS + ZM-HSP17.7 |
| | | | PRO::MO-CRE-EXON1::ST-LS1 |
| | | | INTRON1::MO-CRE EXON2::PINII |
| | | | TERM + OS-UBI TERM + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1::NPTII::SB- |
| | | | UBI TERM + LB |
| 11 | DNA | RV047654 = | RB + LOXP + NOS PRO::ZM- |
| | | PHP104112 | WUS2::IN2-1 TERM + FMV |
| | | | ENH::PCSV ENH::MMV ENH::UBI1ZM |
| | | | PRO::UBI1ZM 5UTR::UBI1ZM |
| | | | INTRON1::ZM-BBM:PROTEIN LINKER |
| | | | 1:AT-CBF1A (MO):OS-T28 TERM + |
| | | | AT-5-IV-2 INS + ZM-HSP17.7 |
| | | | PRO::MO-CRE-EXON1::ST-LS1 |
| | | | INTRON1::MO-CRE EXON2::PINII |
| | | | TERM + OS-UBI TERM + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1::NPTII::SB- |
| | | | UBI TERM + LB |
| 12 | DNA | Os-Actin Pro | OS-ACTIN PRO |
| 13 | DNA | UBI1ZM PRO/5′ | Maize ubiquitin promoter and 5′ |
| | | UTR | untranslated region |
| 14 | DNA | Zm-Bbm | maize Ovule Development Protein2 |
| | | | (maize ortholog of At-BabyBoom), also |
| | | | known as the Bbm gene |
| 15 | PRT | ZM-BBM | Maize ODP2 encoded protein |
| 16 | DNA | Zm-Bbm Motif B | ZM-ODP2 (TR11) or Motif B |
| 17 | PRT | ZM-BBM Motif B | ZM-ODP2 (TR11) or Motif B |
| 18 | DNA | Zm-Bbm Motif A | ZM-ODP2 (TR12) or Motif A |
| 19 | PRT | ZM-BBM Motif A | ZM-ODP2 (TR11) or Motif A |
| 20 | DNA | Zm-Bbm404 | ZM-ODP2 (TR5) or BBM404 |
| 21 | PRT | ZM-BBM404 | ZM-ODP2 (TR5) or BBM404 |
| 22 | PRT | Zemay-BBM2 | Zea mays BBM2 encoded protein |
| 23 | PRT | Zemay-BBM2 | Zea mays BBM2 Motif B encoded |
| | | Motif B | peptide |
| 24 | PRT | Zemay-BBM2 | Zea mays BBM2 Motif A encoded |
| | | Motif A | peptide |
| 25 | PRT | Zemay-BBM2 | Zea mays BBM2 BBM404 encoded |
| | | BBM404 | truncated protein |
| 26 | PRT | Sobic-BBM | Sorghum bicolor BBM encoded protein |
| 27 | PRT | Sobic-BBM Motif | Sorghum bicolor BBM Motif B encoded |
| | | B | peptide |
| 28 | PRT | Sobic-BBM Motif | Sorghum bicolor BBM Motif A encoded |
| | | A | peptide |
| 28 | PRT | Sobic-BBM | Sorghum bicolor BBM BBM404 |
| | | BBM404 | encoded truncated protein |
| 30 | PRT | Sobic-BBM2 | Sorghum bicolor BBM2 encoded |
| | | | protein |
| 31 | PRT | Sobic-BBM2 | Sorghum bicolor BBM2 Motif B |
| | | Motif B | encoded peptide |
| 32 | PRT | Sobic-BBM2 | Sorghum bicolor BBM2 Motif A |
| | | Motif A | encoded peptide |
| 33 | PRT | Sobic-BBM2 | Sorghum bicolor BBM2 BBM404 |
| | | BBM404 | encoded truncated protein |
| 34 | PRT | Misin-BBM | Miscanthus sinensis BBM encoded |
| | | | protein |
| 35 | PRT | Misin-BBM Motif | Miscanthus sinensis BBM Motif B |
| | | B | encoded peptide |
| 36 | PRT | Misin-BBM Motif | Miscanthus sinensis BBM Motif A |
| | | A | encoded peptide |
| 37 | PRT | Misin-BBM | Miscanthus sinensis BBM BBM404 |
| | | BBM404 | encoded truncated protein |
| 38 | PRT | Misin-BBM2 | Miscanthus sinensis BBM2 encoded |
| | | | protein |
| 39 | PRT | Misin-BBM2 Motif | Miscanthus sinensis BBM2 Motif B |
| | | B | encoded peptide |
| 40 | PRT | Misin-BBM2 Motif | Miscanthus sinensis BBM2 Motif A |
| | | A | encoded peptide |
| 41 | PRT | Misin-BBM2 | Miscanthus sinensis BBM2 BBM404 |
| | | BBM404 | encoded truncated protein |
| 42 | PRT | Orsat-BBM | Oryza sativa BBM encoded protein |
| 43 | PRT | Orsat-BBM Motif | Oryza sativa BBM Motif B encoded |
| | | B | peptide |
| 44 | PRT | Orsat-BBM Motif | Oryza sativa BBM Motif A encoded |
| | | A | peptide |
| 45 | PRT | Orsat-BBM | Oryza sativa BBM BBM404 encoded |
| | | BBM404 | truncated protein |
| 46 | PRT | Orsat-BBM2 | Oryza sativa BBM2 encoded protein |
| 47 | PRT | Orsat-BBM2 | Oryza sativa BBM2 Motif B encoded |
| | | Motif B | peptide |
| 48 | PRT | Orsat-BBM2 | Oryza sativa BBM2 Motif A encoded |
| | | Motif A | peptide |
| 49 | PRT | Orsat-BBM2 | Oryza sativa BBM2 BBM404 encoded |
| | | BBM404 | truncated protein |
| 50 | PRT | Orsat-BBM1 | Oryza sativa BBM1 encoded protein |
| 51 | PRT | Orsat-BBM1 | Oryza sativa BBM1 Motif B encoded |
| | | Motif B | peptide |
| 52 | PRT | Orsat-BBM1 | Oryza sativa BBM1 Motif A encoded |
| | | Motif A | peptide |
| 53 | PRT | Orsat-BBM1 | Oryza sativa BBM1 BBM404 encoded |
| | | BBM404 | truncated protein |
| 54 | PRT | Brdis-BBM | Brachypodium distachyon BBM |
| | | | encoded protein |
| 55 | PRT | Brdis-BBM Motif | Brachypodium distachyon BBM Motif B |
| | | B | encoded peptide |
| 56 | PRT | Brdis-BBM Motif | Brachypodium distachyon BBM Motif A |
| | | A | encoded peptide |
| 57 | PRT | Brdis-BBM | Brachypodium distachyon BBM |
| | | BBM404 | BBM404 encoded truncated protein |
| 58 | PRT | Brdis-BBM2 | Brachypodium distachyon BBM2 |
| | | | encoded protein |
| 59 | PRT | Brdis-BBM2 Motif | Brachypodium distachyon BBM2 Motif |
| | | B | B encoded peptide |
| 60 | PRT | Brdis-BBM2 Motif | Brachypodium distachyon BBM2 Motif |
| | | A | A encoded peptide |
| 61 | PRT | Brdis-BBM2 | Brachypodium distachyon BBM2 |
| | | BBM404 | BBM404 encoded truncated protein |
| 62 | PRT | Muaca-Chr2- | Muca acuminata chromosome 2 BBM |
| | | BBM | encoded protein |
| 63 | PRT | Muaca-Chr2- | Muca acuminata chromosome 2 BBM |
| | | BBM Motif B | Motif B encoded peptide |
| 64 | PRT | Muaca-Chr2- | Muca acuminata chromosome 2 BBM |
| | | BBM Motif A | Motif A encoded peptide |
| 65 | PRT | Muaca-Chr2- | Muca acuminata chromosome 2 BBM |
| | | BBM BBM404 | BBM404 encoded truncated protein |
| 66 | PRT | Muaca-Chr9- | Muca acuminata chromosome 9 BBM2 |
| | | BBM2 | encoded protein |
| 67 | PRT | Muaca-Chr9- | Muca acuminata chromosome 9 BBM2 |
| | | BBM2 Motif B | Motif B encoded peptide |
| 68 | PRT | Muaca-Chr9- | Muca acuminata chromosome 9 BBM2 |
| | | BBM2 Motif A | Motif A encoded peptide |
| 69 | PRT | Muaca-Chr9- | Muca acuminata chromosome 9 BBM2 |
| | | BBM2 BBM404 | BBM404 encoded truncated protein |
| 70 | PRT | Sevir-BBM | Setaria viridis BBM encoded protein |
| 71 | PRT | Sevir-BBM Motif | Setaria viridis BBM Motif B encoded |
| | | B | peptide |
| 72 | PRT | Sevir-BBM Motif | Setaria viridis BBM Motif A encoded |
| | | A | peptide |
| 73 | PRT | Sevir-BBM | Setaria viridis BBM BBM404 encoded |
| | | BBM404 | truncated protein |
| 74 | PRT | Sevir-BBM2 | Setaria viridis BBM2 encoded protein |
| 75 | PRT | Sevir-BBM2 Motif | Setaria viridis BBM2 Motif B encoded |
| | | B | peptide |
| 76 | PRT | Sevir-BBM2 Motif | Setaria viridis BBM2 Motif A encoded |
| | | A | peptide |
| 77 | PRT | Sevir-BBM2 | Setaria viridis BBM2 BBM404 encoded |
| | | BBM404 | truncated protein |
| 78 | PRT | Seita-BBM | Setaria italica BBM encoded protein |
| 79 | PRT | Seita-BBM Motif | Setaria italica BBM Motif B encoded |
| | | B | peptide |
| 80 | PRT | Seita-BBM Motif | Setaria italica BBM Motif A encoded |
| | | A | peptide |
| 81 | PRT | Seita-BBM | Setaria italica BBM BBM404 encoded |
| | | BBM404 | truncated protein |
| 82 | PRT | Seita-BBM2 | Setaria italica BBM2 encoded protein |
| 83 | PRT | Seita-BBM2 Motif | Setaria italica BBM2 Motif B encoded |
| | | B | peptide |
| 84 | PRT | Seita-BBM2 Motif | Setaria italica BBM2 Motif A encoded |
| | | A | peptide |
| 85 | PRT | Seita-BBM2 | Setaria italica BBM2 BBM404 encoded |
| | | BBM404 | truncated protein |
| 86 | PRT | Traes-BBM | Triticum aestivum BBM encoded |
| | | | protein |
| 87 | PRT | Traes-BBM Motif | Triticum aestivum BBM Motif B |
| | | B | encoded peptide |
| 88 | PRT | Traes-BBM Motif | Triticum aestivum BBM Motif A |
| | | A | encoded peptide |
| 89 | PRT | Traes-BBM | Triticum aestivum BBM BBM404 |
| | | BBM404 | encoded truncated protein |
| 90 | PRT | Traes-BBM2 | Triticum aestivum BBM2 encoded |
| | | | protein |
| 91 | PRT | Traes-BBM2 | Triticum aestivum BBM2 Motif B |
| | | Motif B | encoded peptide |
| 92 | PRT | Traes-BBM2 | Triticum aestivum BBM2 Motif A |
| | | Motif A | encoded peptide |
| 93 | PRT | Traes-BBM2 | Triticum aestivum BBM2 BBM404 |
| | | BBM404 | encoded truncated protein |
| 94 | PRT | Pahal-BBM | Panicum hallii BBM encoded protein |
| 95 | PRT | Pahal-BBM Motif | Panicum hallii BBM Motif B encoded |
| | | B | peptide |
| 96 | PRT | Pahal-BBM Motif | Panicum hallii BBM Motif A encoded |
| | | A | peptide |
| 97 | PRT | Pahal-BBM | Panicum hallii BBM BBM404 encoded |
| | | BBM404 | truncated protein |
| 98 | PRT | Pahal-BBM2 | Panicum hallii BBM2 encoded protein |
| 99 | PRT | Pahal-BBM2 | Panicum hallii BBM2 Motif B encoded |
| | | Motif B | peptide |
| 100 | PRT | Pahal-BBM2 | Panicum hallii BBM2 Motif A encoded |
| | | Motif A | peptide |
| 101 | PRT | Pahal-BBM2 | Panicum hallii BBM2 BBM404 encoded |
| | | BBM404 | truncated protein |
| 102 | PRT | Metru-BBM | Medicago trunculata BBM encoded |
| | | | protein |
| 103 | PRT | Metru-BBM Motif | Medicago trunculata BBM Motif B |
| | | B | encoded peptide |
| 104 | PRT | Metru-BBM Motif | Medicago trunculata BBM Motif A |
| | | A | encoded peptide |
| 105 | PRT | Metru-BBM | Medicago trunculata BBM BBM404 |
| | | BBM404 | encoded truncated protein |
| 106 | PRT | Vivin-BBM | Vitus vinifera BBM encoded protein |
| 107 | PRT | Vivin-BBM Motif | Vitus vinifera BBM Motif B encoded |
| | | B | peptide |
| 108 | PRT | Vivin-BBM Motif | Vitus vinifera BBM Motif A encoded |
| | | A | peptide |
| 109 | PRT | Vivin-BBM | Vitus vinifera BBM BBM404 encoded |
| | | BBM404 | truncated protein |
| 110 | PRT | Brnap-BBM | Brassica napus BBM encoded protein |
| 111 | PRT | Brnap-BBM Motif | Brassica napus BBM Motif B encoded |
| | | B | peptide |
| 112 | PRT | Brnap-BBM Motif | Brassica napus BBM Motif A encoded |
| | | A | peptide |
| 113 | PRT | Brnap-BBM | Brassica napus BBM BBM404 encoded |
| | | BBM404 | truncated protein |
| 114 | PRT | Cusat-BBM | Cucumis sativa BBM encoded protein |
| 115 | PRT | Cusat-BBM Motif | Cucumis sativa BBM Motif B encoded |
| | | B | peptide |
| 116 | PRT | Cusat-BBM Motif | Cucumis sativa BBM Motif A encoded |
| | | A | peptide |
| 117 | PRT | Cusat-BBM | Cucumis sativa BBM BBM404 encoded |
| | | BBM404 | truncated protein |
| 118 | PRT | Gohir-BBM | Gossypium hirsutum BBM encoded |
| | | | protein |
| 119 | PRT | Gohir-BBM Motif | Gossypium hirsutum BBM Motif B |
| | | B | encoded peptide |
| 120 | PRT | Gohir-BBM Motif | Gossypium hirsutum BBM Motif A |
| | | A | encoded peptide |
| 121 | PRT | Gohir-BBM | Gossypium hirsutum BBM BBM404 |
| | | BBM404 | encoded truncated protein |
| 122 | PRT | Glmax-BBM | Glycine max BBM encoded protein |
| 123 | PRT | Glmax-BBM Motif | Glycine max BBM Motif B encoded |
| | | B | peptide |
| 124 | PRT | Glmax-BBM Motif | Glycine max BBM Motif A encoded |
| | | A | peptide |
| 125 | PRT | Glmax-BBM | Glycine max BBM BBM404 encoded |
| | | BBM404 | truncated protein |
| 126 | DNA | AT-CBF1A | Arabidopsis thaliana CBF1A activation |
| | | | domain gene, encoding aa 116-214 |
| 127 | PRT | AT-CBF1A | Arabidopsis thaliana CBF1A activation |
| | | | domain - aa 116-214 |
| 128 | DNA | AT-CBF31 | Arabidopsis thaliana CBF31 activation |
| | | | domain gene |
| 129 | PRT | AT-CBF31 | Arabidopsis thaliana CBF31 activation |
| | | | domain |
| 130 | DNA | SB-CBF1A | Sorghum bicolor CBF1A activation |
| | | | domain gene |
| 131 | PRT | SB-CBF1A | Sorghum bicolor encoded CBF1A |
| | | | activation domain |
| 132 | DNA | ZM-CBF1A | Zea mays CBF1A activation domain |
| | | | gene |
| 133 | PRT | ZM-CBF1A | Zea mays CBF1A encoded activation |
| | | | domain |
| 134 | DNA | ZM-C1 | Zea mays transcription factor C1 |
| | | | activation domain gene |
| 135 | PRT | ZM-C1 | Zea mays - encoded transcription |
| | | | factor C1 activation domain |
| 136 | DNA | ZM-02 | Zea mays transcription factor O2 |
| | | | activation domain gene |
| 137 | PRT | ZM-O2 | Zea mays - encoded transcription |
| | | | factor O2 activation domain |
| 138 | PRT | ZM-DOF1 | Zea mays DNA binding with One Finger |
| | | | 1 activation domain |
| 139 | PRT | AT-ERF97 | Arabidopsis thaliana Ethylene |
| | | | Responsive Factor 97 activation |
| | | | domain |
| 140 | PRT | AT-ERF98 | Arabidopsis thaliana Ethylene |
| | | | Responsive Factor 98 activation |
| | | | domain |
| 141 | PRT | GM-EDLL-like | Glycine max ERF activation domain |
| | | | with EDLL motif |
| 142 | PRT | MT-EDLL-like | Medicago trunculata ERF activation |
| | | | domain with EDLL motif |
| 143 | PRT | OS-EDLL-like | Oryza sativa ERF activation domain |
| | | | with EDLL motif |
| 144 | PRT | VP16 core | Herpes Simplex Virus VP16 activation |
| | | | core peptide |
| 145 | PRT | AT-CBF1 core | Arabidopsis thaliana CBF1A activation |
| | | | domain with “VP16-like” region |
| 146 | PRT | AT-CBF1m | Arabidopsis thaliana CBF1A activation |
| | | | domain with modified “VP16-like” region |
| 147 | PRT | ZM-DOF1 core | Zea mays DOF1 activation domain with |
| | | | “VP16-like” region |
| 148 | PRT | ZM-DOF1m | Zea mays DFO1 activation domain with |
| | | | modified “VP16-like” region |
| 149 | PRT | AT-DREB1 core | Arabidopsis thaliana DREB1 activation |
| | | | domain with “VP16-like” region |
| 150 | PRT | AT-DREB1m | Arabidopsis thaliana DREB1 activation |
| | | | domain with modified “VP16-like” region |
| 151 | PRT | NT-ERF1 core | Nicotiana tabacum ERF1 activation |
| | | | domain with “VP16-like” region |
| 152 | PRT | NT-ERF1m | Nicotiana tabacum ERF1 activation |
| | | | domain with modified “VP16-like” region |
| 153 | PRT | NT-ERF2 core | Nicotiana tabacum ERF2 activation |
| | | | domain with “VP16-like” region |
| 154 | PRT | NT-ERF2m | Nicotiana tabacum ERF2 activation |
| | | | domain with modified “VP16-like” region |
| 155 | PRT | CR-ORCA core | Catharanthus roseus ORCA activation |
| | | | domain with “VP16-like” region |
| 156 | PRT | CR-ORCAm | Catharanthus roseus ORCA activation |
| | | | domain with modified “VP16-like” region |
| 157 | PRT | LS-PTI4 core | Lycopersicum solanum PTI4 activation |
| | | | domain with “VP16-like” region |
| 158 | PRT | LS-PTI4m | Lycopersicum solanum PTI4 activation |
| | | | domain with modified “VP16-like” region |
| 159 | DNA | VP64 | Herpes Simplex Virus VP64 synthetic |
| 160 | PRT | VP64 | Synthetic multimeric activation domains |
| 161 | DNA | PHP000001 T- | RB + AXIG1 PRO::ZM-WUS2::IN2-1 |
| | | DNA | TERM + ZM-PLTP PRO::ZM- |
| | | | BBM404:CBF1A::OS-T28 TERM + GZ- |
| | | | W64A TERM + SB-ALS PRO:: |
| | | | HRA::SB-PEPC1 TERM + LTP2 |
| | | | PRO::ZS-YELLOW::PINII TERM-LB |
| 162 | DNA | PHP000002 T- | RB + UBI1ZM PRO::BBM404:CBF1A + |
| | | DNA | UBI1ZM PRO::MO-PAT::PINII + LB |
| 163 | DNA | PHP000003 T- | RB + LOXP + HSP17 PRO::CRE + |
| | | DNA | NOS PRO::ZM-WUS2::IN2-1 TERM + |
| | | | ZM-UBI1 PRO::BBM404:CBF1A::ZM- |
| | | | UBI TERM + LOXP + SB-UBI |
| | | | PRO::ZsGREEN1::OS-UBI TERM + SI- |
| | | | ALS PRO::ZM-HRA::SB UBI TERM + |
| | | | LB |
| 164 | PRT | AT-HSFA6B | Activation domain from Arabidopsis |
| | | | thaliana Heat Shock Transcription |
| | | | Factor A6B |
| 165 | PRT | AT-DREB2A | Activation domain from Arabidopsis |
| | | | thaliana DREB2A |
| 166 | PRT | ZM-DOF1 | Activation domain from Zea mays |
| | | | DOF1 DNA-binding with one finger1 |
| | | | gene |
| 167 | DNA | RV048912 = | RB + LOXP + OS-ACTIN PRO::ZM- |
| | | PHP104222 | WUS2::IN2-1 TERM + FMV ENH::PCSV |
| | | | ENH::MMV ENH::UBI1ZM |
| | | | PRO::UBI1ZM 5UTR::UBI1ZM |
| | | | INTRON1::ZM-ODP2::OS-T28 TERM + |
| | | | AT-5-IV-2 INS + |
| | | | ZM-HSP17.7 |
| | | | PRO::MO-CRE-EXON1::ST-LS1 |
| | | | INTRON1::MO-CRE EXON2::PINII |
| | | | TERM + AT-5-IV-2 INS + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1::NPTII::SB- |
| | | | UBI TERM + LB |
| 168 | DNA | RV050602 = | RB + LOXP + UBI1ZM PRO::UBI1ZM |
| | | PHP105696 | 5UTR::UBI1ZM INTRON1::ZM- |
| | | | WUS2::IN2-1 TERM + FMV ENH::PCSV |
| | | | ENH::MMV ENH::UBI1ZM |
| | | | PRO::UBI1ZM 5UTR::UBI1ZM |
| | | | INTRON1::ZM-ODP2::OS-T28 TERM + |
| | | | AT-5-IV-2 INS + ZM-HSP17.7 |
| | | | PRO::MO-CRE-EXON1::ST-LS1 |
| | | | INTRON1::MO-CRE EXON2::PINII |
| | | | TERM + AT-5-IV-2 INS + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1::NPTII::SB- |
| | | | UBI TERM + LB |
| 169 | DNA | RV054309 = | RB + LOXP + NOS PRO::ZM- |
| | | PHP107604 | WUS2::IN2-1 TERM + FMV ENH::PCSV |
| | | | ENH::MMV ENH::UBI1ZM |
| | | | PRO::UBI1ZM 5UTR::UBI1ZM |
| | | | INTRON1::ZM-BBM404:PROTEIN |
| | | | LINKER 1:AT-CBF1A (MO):OS-T28 |
| | | | TERM + AT-5-IV-2 INS + ZM-HSP17.7 |
| | | | PRO::MO-CRE-EXON1::ST-LS1 |
| | | | INTRON1::MO-CRE EXON2::PINII |
| | | | TERM + OS-UBI TERM + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1::NPTII:SB- |
| | | | UBI TERM + LB |
| 170 | DNA | RV052550 = | RB + LOXP + OS-ACTIN PRO::ZM- |
| | | PHP106860 | WUS2::IN2-1 TERM + FMV ENH::PCSV |
| | | | ENH::MMV ENH::UBI1ZM |
| | | | PRO::UBI1ZM 5UTR::UBI1ZM |
| | | | INTRON1::ZM-BBM404:PROTEIN |
| | | | LINKER 1:AT-CBF1A (MO):OS-T28 |
| | | | TERM + AT-5-IV-2 INS + ZM-HSP17.7 |
| | | | PRO::MO-CRE-EXON1::ST-LS1 |
| | | | INTRON1::MO-CRE EXON2::PINII |
| | | | TERM + OS-UBI TERM + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1::NPTII::SB- |
| | | | UBI TERM + LB |
| 171 | DNA | RV052548 = | RB + LOXP + UBI1ZM PRO::UBI1ZM |
| | | PHP106868 | 5UTR::UBI1ZM INTRON1::ZM- |
| | | | WUS2::IN2-1 TERM + FMV ENH::PCSV |
| | | | ENH::MMV ENH::UBI1ZM |
| | | | PRO::UBI1ZM 5UTR::UBI1ZM |
| | | | INTRON1::ZM-BBM404:PROTEIN |
| | | | LINKER 1:AT-CBF1A (MO):OS-T28 |
| | | | TERM + AT-5-IV-2 INS + ZM-HSP17.7 |
| | | | PRO::MO-CRE-EXON1::ST-LS1 |
| | | | INTRON1::MO-CRE EXON2::PINII |
| | | | TERM + OS-UBI TERM + LOXP + SB- |
| | | | UBI PRO::SB-UBI INTRON1::ZS- |
| | | | GREEN1::OS-UBI TERM + SI-UBI1 |
| | | | PRO::SI-UBI1 INTRON1::NPTII::SB- |
| | | | UBI TERM + LB |

### Example 2: Particle Bombardment

Standard protocols for particle bombardment (Finer and McMullen, 1991, In Vitro Cell Dev. Biol.—Plant 27:175-182 and those found in WO2022072335A2) can be used with the methods of the disclosure.

### Example 3: Agrobacterium-Mediated Transformation of Corn

### A. Preparation of Agrobacterium Master Plate.

Agrobacterium tumefaciens harboring a binary donor vector is streaked out from a −80° C. frozen aliquot onto solid 12R medium and cultured at 28° C. in the dark for 2-3 days to make a master plate.

### B. Growing Agrobacterium on Solid Medium.

A single colony or multiple colonies of Agrobacterium are picked from the master plate and streaked onto a second plate containing 810K medium and incubated at 28° C. in the dark overnight.

Agrobacterium infection medium 5 ml) and 100 mM 3′-5′-Dimethoxy-4′-hydroxyacetophenone (acetosyringone; 5 μL) are added to a 14 ml conical tube in a hood. About 3 full loops of Agrobacterium from the second plate are suspended in the tube and the tube was then vortexed to make an even suspension. The suspension (1 ml) is transferred to a spectrophotometer tube and the optical density (550 nm) of the suspension is adjusted to a reading of about 0.35-1.0. The Agrobacterium concentration was approximately 0.5 to 2.0×10⁹ cfu/mL. The final Agrobacterium suspension was aliquoted into 2 mL microcentrifuge tubes, each containing about 1 mL of the suspension. The suspensions were then used as soon as possible.

### C. Growing Agrobacterium on Liquid Medium.

Alternatively, Agrobacterium can be prepared for transformation by growing in liquid medium. One day before infection, a 125 ml flask is prepared with 30 ml of 557A medium and 30 μL spectinomycin (50 mg/mL) and 30 μL acetosyringone (20 mg/mL). A half loopful of Agrobacterium from a second plate is suspended into the flasks and placed on an orbital shaker set at 200 rpm and incubated at 28° C. overnight. The Agrobacterium culture is centrifuged at 5000 rpm for 10 min. The supernatant is removed and the Agrobacterium infection medium with acetosyringone solution is added. The bacteria are resuspended by vortex and the optical density (550 nm) of the Agrobacterium suspension was adjusted to a reading of about 0.35 to 2.0.

### D. Maize Transformation.

Maize seed is surface-sterilized for 15-20 min in 20% (v/v) bleach (5.25% sodium hypochlorite) plus 1 drop of Tween 20 followed by 3 washes in sterile water, germinated and allowed to grow into seedlings for approximately 14 days, and then prepared to produce leaf fragments. Leaf segments are placed in the Agrobacterium infection medium (700A) with 200 μM acetosyringone solution +0.02% Break-Thru® surfactant (Plant Health Technologies, P.O. Box 70013, Boise, ID 83707-0113). The Agrobacterium infection medium is drawn off and 1 ml of the Agrobacterium suspension is added to the leaf segments and allowed to stand for 20 min. The suspension of Agrobacterium and leaf segments is poured through a sterile metal sieve and the liquid is discarded. The leaf segments collected on the metal sieve are transferred using a spatula onto a stack of 3 sterile filter papers, used to wick off excess Agrobacterium-containing liquid, and then a spatula is used to transfer the leaf segments onto a filter paper lying on co-cultivation medium. The plate is incubated in the dark at 21° C. for 1-3 days of co-cultivation.

The filter papers supporting the leaf segments are then transferred to resting medium without selection. Seven days later, the filter papers supporting the leaf segments were transferred to selection medium for three weeks. After selection, healthy growing somatic embryos are transferred using forceps onto maturation medium for two weeks in the dark, at which point the maturation plates are transferred in toto (still containing the maturing somatic embryos) into the light for an additional week. After one week in the light, regenerating plantlets are transferred to rooting medium. After rooting, plantlets are ready for transplanting to the greenhouse.

### Example 4: BBM (ODP2) Truncations

The full-length ZM-ODP2 (BBM) gene (2,133 bp) (SEQ ID Nos: 14 and 15) encoding the full-length wild-type ODP2 protein (710 aa) was truncated to produce three fragments:
ZM-ODP2 (TR11)=Motif “B” which encoded amino acids 60-69 of ZM-ODP2 protein (SEQ ID Nos: 16 and 17)
ZM-ODP2 (TR12)=Motif “A” which encoded amino acids 156-171 of ZM-ODP2 protein (SEQ ID Nos: 18 and 19)
ZM-ODP2 (TR5)=BBM404 which encoded amino acids 266-669 of ZM-ODP2 protein (SEQ ID Nos: 20 and 21)

### Example 5: Fusion of the CBF1A Activation Domain to Truncated Bbm Enhanced Transformation

### (A) Maize Stiff-Stalk Inbred ED85E.

Seeds of maize inbred ED85E were surface sterilized and germinated on medium containing MS salts, sucrose and 2 mg/l ancymidol. Thirteen-day-old seedlings were harvested and the first 3 cm of leaf tissue above the mesocotyl was bisected lengthwise and then mechanically chopped in the blender while suspended in 100 ml of Agrobacterium at an OD of 0.5 (see details in Example 5).

Five T-DNA configurations were tested using 10 seedlings per treatment. After regeneration of TO plantlets and formation of roots (i.e., the stage that plants are typically sent to the greenhouse), leaf tissue from each TO plant was sampled for multiplex PCR to confirm copy number of the integrated T-DNA. All five plasmids contained the same Nos::Wus2 expression cassette and the same 3×ENH:UBI promoter driving expression of the Bbm gene, and experimental results are summarized in Table 2. For all five treatments (5 different Bbm coding sequences), T-DNA delivery as measured by transient Zs-Green1 expression at 3-4 days after infection was good (score=3) to excellent (score=4). In the first control treatment with the full-length Bbm gene (PHP97334), TO plants were recovered at a 600% frequency (e.g., 10 starting seedlings produced 60 TO plants). In the second treatment, the Bbm gene was truncated to include 404 amino acids including the two AP2 DNA-binding domains and the majority of the carboxy-end of the protein (PHP102072), and for this treatment the transformation frequency was reduced relative to the control at 438%. Adding either one copy of the highly conserved amino-peptides “B” and “A” in PHP101977 (as described in WO 2020/214986) or two copies of these motifs (PHP103858) resulted in a further reduction of transformation frequencies, producing values of 113% and 125%, respectively. Surprisingly, when a maize-codon-optimized transcriptional activation domain from the Arabidopsis CBF1A protein (SEQ ID NO: 127) was fused onto the carboxy-end of the truncated B-A-Bbm404 protein (PHP101978), a transformation frequency of 2031% was produced, a roughly 3.4-fold increase above the control full-length Bbm gene.

TABLE 2
Leaf transformation frequencies for maize inbred ED85E using various modified Bbm genes.

| | | | T-DNA | Transformation |
|---|---|---|---|---|
| RV# | Plasmid | Description | Delivery | Frequency |
| RV035126 | PHP97334 | Nos::Wus2 + 3xEnh:Ubi::Bbm (control) | 4 | 600% |
| RV045471 | PHP102072 | Nos::Wus2 + 3xEnh:Ubi::Bbm404 | 3 | 438% |
| RV045472 | PHP101977 | Nos::Wus2 + 3xEnh:Ubi::B-A-Bbm404 | 3.5 | 113% |
| RV045473 | PHP103858 | Nos::Wus2 + 3xEnh:Ubi::B-A-B-A-Bbm404 | 3.5 | 125% |
| RV045474 | PHP101978 | Nos::Wus2 + 3xEnh:Ubi::B-A-Bbm404:CBF1A | 4 | 2031% |

### (B) Maize Non-Stiff-Stalk Inbred GR013D.

Starting with 10 seedlings per replicate, fourteen-day-old seedlings were mechanically chopped in Agrobacterium containing the helper plasmid PHP71539 plus either PHP97334 (full-length wild-type Bbm gene) or PHP101978 (B-A-Bbm404:CBF1A). The experimental treatments were replicated three times (the two plasmid treatments side-by-side) and after selection and plant regeneration to produce TO plantlets, the transformation frequencies were scored. PHP97334 produced transformation frequencies of 50%, 40%, and 10% for a mean and standard deviation of 33%+/−17%. PHP101978 produced transformation frequencies of 210%, 220%, and 180% for a mean and standard deviation of 203%+/−17%. For this inbred, using the truncated Bbm404 fused to the CBF1A activation domain produced over a 6-fold increase in transformation frequency relative to the control plasmid.

(C) Comparing the Full-Length Bbm Gene to the B-A-Bbm404 Truncation, with Each being Fused to Activation Domain CBF1A.

Three plasmids were compared, using Agrobacterium-mediated transformation into maize inbred ED85E leaf tissue, starting with seven seedlings per treatment per replicate. Results for both replicates were consistent, with the full-length Bbm gene and the full-length Bbm fused to CBF1A producing similar transformation frequencies. However, when the truncated B-A-Bbm404 gene was fused to CBF1A, transformation frequency in both replicates was substantially higher in both replicates of the experiment.

TABLE 3
Fusing CBF1A activation domain to the truncated B-A-Bbm404 protein increased
transformation frequencies substantially compared to either the full-length
Bbm gene or the full-length gene fused to the same activation domain.

| | | | Replicate | Replicate |
|---|---|---|---|---|
| | | | 1 Trans- | 2 Trans- |
| | | | formation | formation |
| RV# | Plasmid | Description | Frequency | Frequency |
| RV035126 | PHP97334 | Nos::Wus2 + 3xEnh:Ubi::Bbm (control) | 471% | 329% |
| RV047654 | PHP104112 | Nos::Wus2 + 3xEnh:Ubi::Bbm:CBF1A | 471% | 282% |
| RV045474 | PHP101978 | Nos::Wus2 + 3xEnh:Ubi::B-A-Bbm404:CBF1A | 1389% | 871% |

Similar enhanced transformation frequencies using B-A-Bbm404:CBF1A have been observed in maize inbreds ED85E, GR013D, and GR0112. In addition, improved transformation has also been observed in the tropical maize inbreds JFDYY, EEP7E, and EENW5.

(D) Comparing the Bbm404 Truncation to the B-A-Bbm404 Truncation, with Each being Fused to Activation Domain CBF1A.

Maize inbred GR0112 leaf tissue was transformed with Agrobacterium harboring the helper plasmid PHP71539 plus either PHP97334 (Nos::Wus2, full length Bbm and no activation domain), PHP104222 (Act::Wus2, full length Bbm and no activation domain), PHP105696 (Ubi::Wus2, full length Bbm and no activation domain), PHP101978 (Nos::Wus2, B-A-Bbm404:CBF1A), PHP104187 (Act::Wus2, B-A-Bbm404:CBF1A), PHP107604 (Nos::Wus2, Bbm404:CBF1A), PHP106860 (Act::Wus2, Bbm404:CBF1A), or PHP106868 (Ubi::Wus2, Bbm404:CBF1A). Results of these transformations are shown in Table 4. GR0112 displayed lower transformation frequency with PHP97334, but showed increased transformation frequency with PHP105696 (Ubi::Wus2). Constructs with B-A-Bbm404:CBF1A transformed GR0112 at double the transformation frequency compared to the control PHP97334. In the absence of the B-A domains, with either Nos::Wus2 or Act::Wus2, the transformation frequency decreased 3-5 folds compared the PHP97334. However, PHP106868 with Ubi::Wus2 and Bbm404:CBF1A gave the highest transformation frequency of 160%.

TABLE 4
Comparison of transformation frequencies in maize inbred
GR0112 between full-length Bbm gene and truncated Bbm404
or B-A-Bbm404 proteins with CBF1A activation domain fused

| | | | Transformation |
|---|---|---|---|
| RV# | Plasmid | Description | Freq (%) |
| RV035126 | PHP97334 | Nos::Wus2 + 3XEnh:Ubi::Bbm (control) | 30% |
| RV048912 | PHP104222 | Act::Wus2 + 3XEnh:Ubi::Bbm | 56% |
| RV050602 | PHP105696 | Ubi::Wus2 + 3XEnh:Ubi::Bbm | 117% |
| RV045474 | PHP101978 | Nos::Wus2 + 3XEnh:Ubi::B-A-Bbm404:CBF1A | 62% |
| RV048557 | PHP104187 | Act::Wus2 + 3XEnh:Ubi::B-A-Bbm404:CBF1A | 60% |
| RV054309 | PHP107604 | Nos::Wus2 + 3XEnh:Ubi::Bbm404:CBF1A | 6% |
| RV052550 | PHP106860 | Act::Wus2 + 3XEnh:Ubi::Bbm404:CBF1A | 10% |
| RV052548 | PHP106868 | Ubi::Wus2 + 3XEnh:Ubi::Bbm404:CBF1A | 160% |

### Example 6: Use of B-A-Bbm404:CBF1A Results in Enhanced Transformation Frequencies in Other Cereal Crops

Comparing transformation frequencies after Agrobacterium transformation with the control plasmid PHP97334 (WT Bbm gene) or PHP101978 (B-A-Bbm404:CBF1A) in leaf tissue from Setaria italica (foxtail millet) resulted in transformation frequencies of 40% for PHP97334 (8 TO plants from 20 starting seedlings) or an increased transformation frequency of 190% (19 TO plants from 10 starting seedlings) for PHP97334. These relative transformation frequencies for Setaria italica are reflected in the scores of + (single +) for plasmid RV035126, also known as PHP973343) and a score of +++ (triple plus) for plasmid RV045474, also known as PHP101978 in Table 5 below. For pearl millet, a higher transformation score than the comparable plasmid containing simply the full-length Bbm gene was also observed. Testing in barley and wheat are in progress, and it is anticipated that the B-A-Bbm404:CBF1A treatment will also result in higher transformation frequencies than the unmodified Bbm treatment.

TABLE 5
Transformation scores for various Poaceae crops using either full-length
Bbm or the truncated B-A-Bbm404 fused to CBF1A activation domain.

| | | | Nos:wus + 3xEnh | Act:wus + 3xEnh |
|---|---|---|---|---|
| | Nos:wus + 3xEnh | Act:wus + 3xEnh | ubi:B-A-Bbm404- | ubi:B-A-Bbm404- |
| | ubi:Bbm | ubi:Bbm | CBF1A | CBF1A |
| Monocot plant | RV035126 | RV048848 | RV045474 | RV048557 |
| Barley | ++ | +++ | NT | NT |
| Setaria italica | + | + | +++ | NT |
| Peal Millet | + | + | ++ | ++ |
| Wheat 25R61 | − | + | − | + |

NT = not yet tested.

### Example 7. Screening Different BBM Orthologous Sequences and Different Activation Domains Demonstrated a Wide Range of Components that Improved Transformation

To assess both the range of orthologous BBM proteins that can be used to first create the three optimal BBM-component fragments (i.e. the A and B motifs and the larger BBM404-like fragments) and then combined with a heterologous activation domain for improved Poaceae explant transformation, PHP101978 was used in an Agrobacterium-mediated leaf transformation assay.
(A) Different activation domains (VP64, ERF2, CBF3I, PTI4, PTI4m, DREB1A, DREB1Am, VP16, DOF1, HSFA6B, DREB2A corresponding to SEQ ID NO: 160, SEQ ID NO: 153, SEQ ID NO: 128, SEQ ID NO: 157, SEQ ID NO: 158, SEQ ID NO: 149, SEQ ID NO: 150, SEQ ID NO: 144, SEQ ID NO: 166, SEQ ID NO: 164, SEQ ID NO: 165, respectively) were tested using the expression cassette configuration found in PHP101978, maintaining all molecular components except for the heterologous activation domain fused to the carboxy-end of Zm-Bbm404. The T-DNA is composed of RB+Nos::Wus2+3×ENH:UBI PRO:B-A-BBM404: [ACT]+Sb-UBI PRO::ZsGreen1+Si-UBI PRO::NPTII, where [ACT] represents the different activation domains that were substituted for At-CBF1A in the construct PHP101978.

To facilitate convenient transformation schedules, the activation domain constructs were split into three groups. The constructs PHP101978 and PHP97334 were included with every group for comparison of transformation frequencies within that experiment. Transformations were done in maize inbred ED85E leaf tissue, starting with 5 seedlings per treatment per replicate. Each experiment had three replicates. The results of all experiments with three replicates combined are given in Table 6.

TABLE 6
Fusing different activation domains to the truncated B-A-Bbm404 protein
increased transformation frequencies substantially compared to the
full-length Bbm without a heterologous activation domain.

| | Vector | Transformation Freq (%) |
|---|---|---|
| Group 1 | PHP97334 (Full length BBM, | 160% |
| | no heterologous activation | |
| | domain, SEQ ID NO: 4) | |
| | PHP101978 (CBF1A, SEQ ID | 321% |
| | NO: 8) | |
| | VP64 (SEQ ID NO: 160) | 455% |
| | ERF2 (SEQ ID NO: 153) | 301% |
| | CBF3I (SEQ ID NO: 129) | 231% |
| Group 2 | PHP97334 (Full length BBM, | 287% |
| | no heterologous activation | |
| | domain, SEQ ID NO: 4) | |
| | PHP101978 (CBF1A, SEQ ID | 460% |
| | NO: 8) | |
| | PTI4 (SEQ ID NO: 157) | 517% |
| | PTI4m (SEQ ID NO: 158) | 299% |
| | DREB1A (SEQ ID NO: 149) | 287% |
| | DREB1Am (SEQ ID NO: 150) | 448% |
| Group 3 | PHP97334 (Full length BBM, | 254% |
| | no heterologous activation | |
| | domain, SEQ ID NO: 4) | |
| | PHP101978 (CBF1A, SEQ ID | 522% |
| | NO: 8) | |
| | VP16 (SEQ ID NO: 144) | 558% |
| | DOF1 (SEQ ID NO: 166) | 283% |
| | HSFA6B (SEQ ID NO: 164) | 232% |
| | DREB2A (SEQ ID NO: 165) | 304% |

Activation domains across both monocots and dicot plant species will work in the method. Candidate heterologous activation domains from protein orthologs from various exemplary plant species (Arabidopsis, Brassica, soybean, maize, rice, sorghum, etc.) are tested for a range of different transcription factors, including CBF1A, CBF3I, CBF1E, ERF98, ERF1, DOF1, ORCA, PTI4, C1, OP2, ARF, LFY, LEC1, LEC2, MADS, bHLH, bZIP, HBP-1a, WRKY, NAM, CUC genes. For example, when the activation domains from At-CBF1A and At-CBF3I were fused to B-A-BBM404, it was demonstrated that CBF1A produced a strong stimulation of somatic embryo formation from leaf tissue, and that CBF31 produced a similar strong response (Table 6). It is expected that the maize C1 activation domain will also produce a slightly decreased response relative to CBF31 or CBFA1. In contrast, using the At-CBF1E or Zm-02 activation domains will produce increasingly weaker somatic embryo stimulation. Thus, it is envisaged that this assay can be used to identify activation domains for use in the method that will produce a range of somatic embryo stimulation from weak responses (Zm-02) to gradually increasing responses (CBF1E, then C1), and finally strong somatic embryo stimulation as with CBF1A and CBF31. Activation domains share similar properties across eukaryotic cells, and thus activation domains such as the Herpes Simplex Virus VP16 (and the synthetic multimeric form VP64) worked well in the method when fused to B-A-BBM404 (Table 6).
(B) Modified activation domains that increase or decrease the strength of activation can also be used in the method. Using the wild-type activation domain sequences and modified amino-acid sequences described by Li et al., 2013 (Plant Biotechnology Journal, 11, 671-680) fused to B-A-BBM404 will work to stimulate somatic embryo formation and transformation frequencies. Thus, use of CBF1, DOF1, DREB1, ERF1, ERF2, ORCA, and PTI4 transactivation domains as well as these same domains that have been modified to have a stronger similarity to the VP16 core transactivation domain are all anticipated to be useful in stimulating leaf somatic embryo formation and increasing transformation frequencies. Among these domains, the DREB1A and DREB1Am (where “m” stands for modified sequence as described by Li et al) showed strong stimulation of leaf somatic embryo formation and transformation (Table 6).
(C) As demonstrated with the core activation domain of VP16 that was multimerized to produce VP64 with greatly increased activation levels (Table 6), using either homomultimers (i.e. CBF1A:CBF1A:etc) or heteromultimers (i.e. CBF1A:ERF2m:PTI4) where the multimeric string (2×, 3×, 4×, etc.) is attached to a single B-A-BBM404 protein will increase the strength of transactivation of endogenous Bbm target genes resulting in a proportionally greater stimulation of somatic embryo growth and increased transformation frequency.
(D) Using the same plasmid design for testing (PHP101978) but substituting Bbm paralogs or orthologs will result in similar positive results. Thus, Bbm, or Bbm1, or Bbm2 proteins from different plant species can be truncated in a similar manner and assembled in the same fashion (B-A-BBM404) and fused to an exemplary activation domain such as CBF1A, and when cloned behind the 3×ENH: UBI PRO will be expected to produce similar enhanced leaf transformation frequencies (see SEQ ID Nos: xx-yy).
(E) Different viral enhancer elements with a different strong constitutive promoter in place of the maize UBI PRO also work in the invention.

### Example 8. Use of BBM404:CBF1A and/or B-A-BBM404:CBF1A Improves Immature Embryo Transformation in Recalcitrant Inbreds

The following experiment demonstrates that expression of BBM404:CBF1A (or B-A-BBM404:CBF1A) and WUS2 immediately after Agrobacterium infection results in direct somatic embryogenesis and regeneration in the recalcitrant public maize inbred W22.

The PLTP promoter driving BBM404:CBF1A and the AXIG1 promoter driving WUS2 expression results in rapid, direct somatic embryo formation after transformation of maize immature embryos.

Immature embryos (2-2.5 mm in length) are harvested from public maize inbred W22 approximately 11 days after pollination, and are infected with Agrobacterium strain LBA4404 THY-TD-containing the helper plasmid PHP71539 and a T-DNA with the following composition; RB+AXIG1 PRO::ZM-WUS2::IN2-1 TERM+ZM-PLTP PRO::ZM-BBM404:CBF1A::OS-T28 TERM+GZ-W64A TERM+SB-ALS PRO::HRA::SB-PEPC1 TERM+LTP2 PRO::ZS-YELLOW::PINII TERM-LB (PHP000001). Agrobacterium is grown on solid medium overnight, and then suspended to an optical density of 0.5 (at 520 nm) and the immature embryos are incubated in the Agrobacterium suspension for 5 minutes before removal from the liquid to be placed on solid 7101 medium at 21° C. overnight.

After 24 hours, the embryos are moved to 605T medium to begin selection against the Agrobacterium. After 6 days, numerous small somatic embryos are observed on the surface of each of the 124 treated immature embryos. Each immature embryo contained numerous, distinct, individual somatic embryos; many being supported on clearly defined suspensors.

Seven days after Agro-infection, the embryos are transferred to maturation medium (289Q medium+0.1 mg/l imazapyr), using the imidazolinone herbicide to select for transgenic embryos. After 14 days on the maturation medium, the mature embryos are moved onto rooting medium (13158H medium; 13158 medium plus 25 mg/l cefotaxime) and leaf pieces are sampled for PCR analysis. Herbicide-resistant plants are PCRed and sent to the greenhouse between 32-34 days after the beginning of the experiment (when the Agrobacterium transformation is started). Plants are sampled for PCR by taking two samples from each plant, one from each of two opposing ears (from opposite sides of the plant) to check for the possibility of any of the plants being only partially transformed (chimeric). PCR results for each pair of samples from all the plants are consistent with other, indicating that no chimeric plants are produced, and that the TO plants are homogenously transgenic. It is expected that expression of BBM404:CBF1A plus Wus2 will result in efficient production of somatic embryos after transformation of immature embryos in W22, resulting in rapid production of high numbers of transgenic TO plants.

B. Use of BBM404:CBF1A alone improves immature embryo transformation of Pioneer Stiff-Stalk Inbred PHP38.

In previous studies, Agrobacterium-mediated transformation immature embryo using Nos::Wus2 plus Ubi::Bbm in Pioneer Stiff-Stalk inbred PHP38 has resulted in transformation frequency of over 50%, while use of Ubi::Bbm alone produced a 10% frequency (Lowe et al., 2016, Plant Cell 28:1998-2015). Using immature embryos from this same inbred PHP38 for transformation using Agrobacterium to deliver a T-DNA containing Ubi::BBM404:CBF1A (PHP000002), it is expected that high transformation frequencies above 50% will be observed.

Also in Lowe et al (2016, Plant Cell 28:1958-1998-2015) it was demonstrated that use of Nos::Wus2 plus Ubi::Bbm could be used to recover transgenic embryogenic calli from the embryo axis of mature seed after Agrobacterium transformation. When BBM404:CBF1A is substituted for Bbm in such a construct (PHP000003), it is expected that mature-seed-derived embryo tissue will produce much higher frequencies of transgenic embryogenic calli and T0 plants.

What is claimed is:

1. A polynucleotide encoding a recombinant transcription factor, the recombinant transcription factor comprising a nucleic acid binding domain and a transcriptional activation domain,
wherein the nucleic acid binding domain comprises a Bbm truncated polypeptide, and the nucleic acid binding domain is capable of binding a gene regulatory sequence;
wherein the transcriptional activation domain comprises a transcriptional activator polypeptide, wherein the transcriptional activation domain is capable of activating transcription of a target gene;
wherein the nucleic acid binding domain comprises at least 50 amino acid residues, and the transcriptional activation domain comprises at least 20 amino acid residues; and
wherein the nucleic acid binding domain and the transcriptional activation domain do not naturally occur in the same polypeptide.

2. The polynucleotide of claim 1, wherein the nucleic acid binding domain comprises a Bbm truncated polypeptide operably linked to (i) a Bbm A polypeptide, (ii) a Bbm B polypeptide, or (iii) both a Bbm A polypeptide and a Bbm B polypeptide.

3. The polynucleotide of claim 1, wherein the nucleic acid binding domain does not comprise a Bbm A polypeptide or a Bbm B polypeptide.

4. The polynucleotide of claim 1, wherein the Bbm truncated polypeptide comprises at least 90% amino acid sequence identity to any one of SEQ ID NO: 21, 25, 29, 33, 37, 41, 45, 49, 53, 57, 61, 65, 69, 73, 77, 81, 85, 89, 93, 97, 101, 105, 109, 113, 117, 121, or 125.

5. The polynucleotide of claim 2, wherein the Bbm B polypeptide comprises any one of SEQ ID NO: 17, 23, 27, 31, 35, 39, 43, 47, 51, 55, 59, 63, 67, 71, 75, 79, 83, 87, 91, 95, 99, 103, 107, 111, 115, 119, or 123 or any otherwise identical sequence where a single amino acid substitution, insertion, or deletion has been made.

6. The polynucleotide of claim 2, wherein the Bbm A polypeptide comprises any one of SEQ ID NO: 19, 24, 28, 32, 36, 40, 44, 48, 52, 56, 60, 64, 68, 72, 76, 80, 84, 88, 92, 96, 100, 104, 108, 112, 116, 120, or 124 or any otherwise identical sequence where a single amino acid substitution, insertion, or deletion has been made.

7. The polynucleotide of claim 1, wherein the transcriptional activator polypeptide comprises at least 90% amino acid sequence identity to any one of SEQ ID NO: 127, 129, 131, 133, 135, 137-158, 160, or 164-166.

8. The polynucleotide of claim 1, wherein the transcriptional activator polypeptide comprises a CBF1A polypeptide.

9. The polynucleotide of claim 1, wherein the transcriptional activator polypeptide comprises at least 90% amino acid sequence identity to SEQ ID NO: 127.

10. The polynucleotide of claim 1, wherein the transcriptional activator polypeptide comprises a CBF3I polypeptide.

11. The polynucleotide of claim 1, wherein the transcriptional activator polypeptide comprises at least 90% amino acid sequence identity to SEQ ID NO: 129.

12. The polynucleotide of claim 1, wherein the transcriptional activation domain comprises multiple transcriptional activator polypeptides.

13. The polynucleotide of claim 12, wherein the transcriptional activation domain comprises two CBF1A polypeptides, two CBF3I polypeptides, or one CBF1A polypeptide and one CBF31 polypeptide.

14. The polynucleotide of claim 1, wherein the nucleic acid binding domain comprises a Bbm B polypeptide, a Bbm A polypeptide, and a Bbm truncated polypeptide and
wherein the transcriptional activator polypeptide comprises a CBF1A polypeptide.

15. The polynucleotide of claim 14, wherein the Bbm B polypeptide comprises SEQ ID NO: 17, the Bbm A polypeptide comprises SEQ ID NO: 19, and the Bbm truncated polypeptide comprises at least 90% amino acid sequence identity to SEQ ID NO: 21 and
wherein the CBF1A polypeptide comprises at least 90% amino acid sequence identity to SEQ ID NO: 127.

16. The polynucleotide of claim 1, wherein the nucleic acid binding domain comprises a Bbm truncated polypeptide and
wherein the transcriptional activator polypeptide comprises a CBF1A polypeptide.

17. The polynucleotide of claim 16, wherein the Bbm truncated polypeptide comprises at least 90% amino acid sequence identity to SEQ ID NO: 21 and
wherein the CBF1A polypeptide comprises at least 90% amino acid sequence identity to SEQ ID NO: 127.

18. The recombinant transcription factor encoded by the polynucleotide of claim 1.

19. A method of producing a recombinant monocot plant, the method comprising:
contacting a monocot plant cell with a first polynucleotide encoding a gene of interest, wherein the gene of interest is heterologous to the monocot plant cell;
contacting the monocot plant cell with a second polynucleotide encoding the recombinant transcription factor of claim 18;
selecting a monocot plant cell that has incorporated the gene of interest into its genome; and
regenerating a recombinant monocot plant from the selected monocot plant cell.

20. The method of claim 19, further comprising contacting the monocot plant cell with a third polynucleotide encoding a functional Wuschel or Wuschel homeobox (WUS/WOX) polypeptide.
